(19) United States
(12) Patent Application Publication
(10) Pub. No.: US 2020/0172584 A1
(43) Pub. Date: Jun. 4, 2020

(54) COMPOSITION AND METHOD COMPRISING ZEIN PROTEIN

(71) Applicant: TECHNION RESEARCH & DEVELOPMENT FOUNDATION LIMITED, Haifa (IL)

(72) Inventors: Ayelet FISHMAN, Haifa (IL); Ilil DAVIDESKO-VARDI, Kfar Masaryk (IL); Sivan ISASCHAR-OVDAT, Netanya (IL); Jovana GLUSAC, Haifa (IL)

(21) Appl. No.: 16/631,561

(22) PCT Filed: Jul. 16, 2018
(86) PCT No.: PCT/IL2018/050785

§ 371 Date: Jan. 16, 2020

Related U.S. Application Data
(60) Provisional application No. 62/533,068, filed on Jul. 16, 2017.

(51) Int. Cl. C07K 14/425; A23L 33/185; A23J 3/14; A23J 1/12

(52) U.S. Cl. CPC C07K 14/425; A23L 33/185; A23V 2002/00; A23J 1/12; A23J 3/14

(57) ABSTRACT

Compounds and compositions comprised of zein protein and a polypeptide of a protein extract, wherein the zein protein and the polypeptide of a protein extract are linked to each other are disclosed. Process of preparing the compounds compositions is also disclosed. Uses of the compositions as a nutritional supplement is also disclosed.

### CROSS REFERENCE TO RELATED APPLICATIONS

This application claims the benefit of priority to U.S. Provisional Patent Application No. 62/533,068 filed Jul. 16, 2017, the contents of which are incorporated herein by reference in their entirety.

### FIELD OF THE INVENTION

The present invention is directed to; inter alia, compositions comprising zein protein and a polypeptide of a of a protein extract and methods of manufacturing and using said compositions as a nutritional supplement.

### BACKGROUND OF THE INVENTION

One of the most pressing challenges related to the world's growing population is high food consumption, particularly animal proteins, which are predicted to double in their demand by 2050. Thus, there is an urgent need in increasing supply from new and sustainable protein sources. Plant proteins has become a current research hotspot and have potential to complement or replace animal proteins in various food applications. Particularly, plant proteins extracted from waste have been recognized as promising raw materials for sustainable production of new protein-rich food ingredients. Zein, the major corn protein, is typically isolated from corn gluten meal (CGM), a protein-rich co-product of corn wet mills.

The major applications of zein as a polymer material for film, coatings and plastics. Zein is mainly rich in glutamic acid (21-26%), leucine (20%), proline (10%), and alanine (10%), but lacks the essential amino acids lysine and tryptophan. Therefore, zein has a poor nutritional value, which is consequently considered unacceptable in human food products.

Enzymatic technologies applied to plant protein rich fractions, concentrates or isolates provides a safe biological technique which can be used to obtain products with desirable properties.

### SUMMARY OF THE INVENTION

The present invention, in some embodiments thereof, relates to compounds and compositions comprising zein, and methods for zein protein functionalization, and uses the same for e.g., nutritional applications. In some embodiments, the zein protein is selected from the group consisting of: alpha-zein, beta-zein, gamma-zein, and delta-zein.

According to some embodiments, there is provided a compound comprising a zein protein and a polypeptide of a protein extract, wherein the zein protein and said polypeptide of a protein extract are linked to each other.

In some embodiments, the zein protein and the polypeptide of a protein extract are linked to each other via a covalent bond. In some embodiments, the covalent bond comprises at least one tyrosine moiety. In some embodiments, the covalent bond comprises at least one tyrosine moiety covalently bonded to a moiety selected from the group consisting of lysine, tyrosine and cysteine.

According to some embodiments, there is provided a composition comprising a plurality of compounds of the invention, in the form of an agglomerate of particles. In some embodiments, at least 25% of the plurality of particles are characterized by a size in the range of 15 micrometers to 350 micrometers.

In some embodiments, the composition is in the form of oil-in-water emulsion. In some embodiments, the composition is the in the form of a gel. In some embodiments, the composition is the in the form of a paste. In some embodiments, the emulsion is characterized by a storage modulus of at least 200 Pa. In some embodiments, the emulsion comprises 5% to 50% (w/w) oil. In some embodiments, the composition is for use in nutritional supplementation. In some embodiments, the composition is identified for use for nutritional supplementation.

According to some embodiments, there is provided a composition comprising an oil-in-water emulsion, wherein the emulsion comprises a zein protein and a polypeptide of a protein extract, and 1% to 80% of oil, by weight. In some embodiments, the oil is selected from olive oil, corn oil, coconut oil, or a combination thereof. In some embodiments, the oil is olive oil.

In some embodiments, the composition further comprises a cross-linking enzyme. In some embodiments, the enzyme is tyrosinase. In some embodiments, the compounds of the invention within composition are covalently linked by tyrosinase. In some embodiments, the tyrosinase is derived from Bacillus megaterium. In some embodiments, the enzyme is characterized by an enzymatic activity in a condition selected from: pH in the range of from 5 to 11.5 and a temperature in the range of from 20 to 65° C.

In some embodiments, the concentration of the zein protein is in the range of from 0.1% to 20% (w/w). In some embodiments, the concentration of the zein protein is in the range of from 0.1% to 10% (w/w). In some embodiments, the concentration of the polypeptide of a protein extract is in the range of from 0.1% to 40% (w/w). In some embodiments, the concentration of the polypeptide of a protein extract is in the range of from 1% to 25% (w/w). In some embodiments, the concentration of the tyrosinase in the range of from 0.05% to 0.5% (w/w).

In some embodiments, the concentration of the zein protein is in the range of from 0.1% to 20% (w/w), the concentration of the polypeptide of a protein extract is in the range of from 0.1% to 40% (w/w) and the concentration of the tyrosinase in the range of from 0.05% to 0.5% (w/w).

In some embodiments, the concentration of the zein protein is in the range of from 0.1% to 10% (w/w), the concentration of the polypeptide of a protein extract is in the range of from 1% to 25% (w/w) and the concentration of the tyrosinase in the range of from 0.05% to 0.5% (w/w). In some embodiments, the composition further comprising an enzymatic cross-linking mediator.

According to some embodiments, there is provided a process for covalently linking a zein protein and a polypeptide of a protein extract, the process comprising the steps: (a) mixing a solution comprising a zein protein, a solution comprising a polypeptide of a protein extract, and a solution comprising tyrosinase thereby creating a mixture thereof; and (b) incubating the mixture for at least 1 h at 25-60° C., thereby covalently linking the zein protein and the polypeptide of the protein extract.

According to some embodiments, there is provided a process for providing a nutritional supplementation, the process comprising the steps: (a) mixing a solution comprising a zein protein, a solution comprising a polypeptide of a protein extract, and a solution comprising tyrosinase thereby creating a mixture thereof; and (b) incubating the mixture for at least 1 h at 25-60° C., thereby covalently linking the zein protein and the polypeptide of the protein extract, thereby providing the nutritional supplementation.

According to some embodiments, there is provided a compound comprising a polypeptide derived from zein protein and an organic molecule comprising a moiety selected from the group consisting of primary amine, phenol and thiol, or any combination thereof. In some embodiments, the polypeptide and the organic molecule are covalently linked to each other via a covalent bond. In some embodiments, the covalent bond comprises at least one phenolic moiety. In some embodiments, the phenolic moiety is covalently bonded to a moiety selected from the group consisting of primary amine, phenol and thiol. In some embodiments, the organic molecule is selected from the group consisting of protein, polysaccharides and polynucleic acids. In some embodiments, the organic molecule is a therapeutic agent.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is an image of sodium dodecyl sulfate polyacrylamide gel electrophoresis (SDS-PAGE) patterns of tyrosinase derived from Bacillus megaterium (TyrBm)-polymerized zein. M—Molecular size marker. Lane 1-7: zein with TyrBm at 0, 30, 90, 120, 180, 240 and 300 min, respectively. Lane 8-9: zein without TyrBm at 0 and 300 min, respectively. Lane 10-12: TyrBm without zein at 0, 120 and 240 min, respectively.

FIG. 2 is an image of the non-crosslinked and TyrBm-crosslinked zein stabilized o/w emulsion after 4 hours of incubation (o/w: oil in water).

FIG. 3 is an image of SDS-PAGE patterns of TyrBm-polymerized potato protein (PP) and zein stabilized emulsion; M—Molecular size marker; Lane 1: non-crosslinked emulsion; Lane 2: crosslinked emulsion; Lane 3: PP fraction; Lane 4: zein fraction.

FIG. 4 is an image of a visible observation of non-crosslinked (vial A) and TyrBm-crosslinked PP+zein (vial B) stabilized o/w emulsion over one month of storage at room temperature.

FIG. 5 is a graph of particle size distribution of oil droplets in the non-crosslinked and TyrBm-crosslinked zein-PP stabilized o/w emulsion after 4 h of crosslinking. Emulsions were diluted with water in the absence of 1% SDS (solid lines) and in the presence of 1% SDS (dashed lines).

FIGS. 6A-B are graphs of the photocentrifuge transmission profiles as a function of length of sample cuvettes of the non-crosslinked (FIG. 6A) and TyrBm-crosslinked PP+zein stabilized o/w emulsion (FIG. 6B). Images of the cuvettes after centrifugation are presented on the right of each figure. Arrows indicate movement of the transmission profiles as a function of time.

FIGS. 7A-B are graphs of apparent viscosity (FIG. 7A) and frequency sweep (FIG. 7B) of non-crosslinked and TyrBm-crosslinked zein-PP stabilized o/w emulsion after 4 h of incubation.

FIGS. 8A-B are images of inverted micrographs of 3 h o/w emulsions stabilized with non-crosslinked PP+zein (FIG. 8A) and TyrBm-crosslinked PP+zein (FIG. 8B) that were crosslinked for 4 h. Bars are 10 micrometers.

FIG. 9 is an image of SDS-PAGE pattern of soluble pea protein concentrate; M-Molecular size marker; lane 1: pea protein concentrate (pH 10).

FIGS. 10A-B are images of SDS-PAGE patterns of TyrBm-polymerized pea protein; M—Molecular size marker; Lane 1-6: pea protein with TyrBm at 0, 30, 60, 120, 180 and 240 min, respectively; Lane 7-8: pea protein without TyrBm at 0 and 240 min, respectively (FIG. 10A) and SDS-PAGE patterns of TyrBm-polymerized pea and zein protein; M-Molecular size marker; Lane 1-6: pea protein and zein with TyrBm at 0, 30, 60, 120, 180 and 240 min, respectively; Lane 7-8: pea protein and zein without TyrBm at 0 and 240 min, respectively (FIG. 10B). The arrow indicates the TyrBm band.

FIGS. 11A-B are images of visible observation of non-crosslinked and TyrBm-crosslinked pea protein stabilized o/w emulsions (FIG. 11A) and non-crosslinked and TyrBm-crosslinked zein and pea protein stabilized o/w emulsion over one month of storage at the room temperature (FIG. 11B).

FIGS. 12A-B are images of SDS-PAGE profiles under reducing conditions of adsorbed protein fractions in non-crosslinked and TyrBm-crosslinked pea protein emulsion; Lanes: coarse emulsion (1 and 5), fine emulsion (2 and 6), after 1 h of incubation (3 and 7), after 2 h of incubation (4 and 8); M—Molecular size marker (FIG. 12A) and SDS-PAGE profiles under reducing conditions of adsorbed protein fractions in non-crosslinked and TyrBm-crosslinked pea-zein emulsion; Lanes: coarse emulsion (1 and 5), fine emulsion (2 and 6), after 1 h of incubation (3 and 7), after 2 h of incubation (4 and 8); M—Molecular size marker (FIG. 12B).

FIG. 13 is an image of SDS-PAGE profiles under reducing conditions of non-adsorbed protein fractions in non-crosslinked and TyrBm-crosslinked pea stabilized emulsion; Lanes: pea supernatant (1 and 7), after TyrBm (or buffer) addition (2 and 8), coarse emulsion (3 and 9), fine emulsion (4 and 10), after 1 h of incubation (5 and 11), after 2 h of incubation (6 and 12).

FIG. 14 is an image of SDS-PAGE profiles under reducing conditions of non-adsorbed protein fractions in non-crosslinked and TyrBm-crosslinked pea-zein emulsion; Lanes: pea supernatant (1 and 7), after buffer (TyrBm) addition (2 and 8), coarse emulsion (3 and 9), fine emulsion (4 and 10), after 1 h of incubation (5 and 11), after 2 h of incubation (6 and 12).

FIGS. 15A-B present apparent viscosity of pea protein stabilized non-crosslinked and TyrBm-crosslinked o/w emulsion (FIG. 15A) and mixture of zein and pea protein stabilized non-crosslinked and TyrBm-crosslinked o/w emulsion (FIG. 15B) after 2 hours of incubation.

FIGS. 16A-B are graphs of particle size distribution of oil droplets in pea protein stabilized o/w emulsion (FIG. 16A) and zein and pea protein stabilized o/w emulsion (FIG. 16B) after 2 hours of crosslinking: the non-crosslinked and TyrBm-crosslinked emulsion. Emulsions were diluted with water in the absence of 1% SDS (solid lines) and in the presence of 1% SDS (dashed lines).

FIGS. 17A-B are graphs of frequency sweep of pea protein stabilized non-crosslinked and TyrBm-crosslinked o/w emulsion (FIG. 17A) and mixture of zein and pea protein stabilized non-crosslinked and TyrBm-crosslinked o/w emulsion (FIG. 17B) after 2 hours of incubation.

FIGS. 18A-D are images of inverted micrographs of pea protein stabilized non-crosslinked (FIG. 18A) and TyrBm-crosslinked o/w emulsion (FIG. 18B) and mixtures of zein and pea protein stabilized non-crosslinked (FIG. 18C) and TyrBm-crosslinked o/w emulsion (FIG. 18D) after 2 hours of incubation; Bars are 10 μm; Micrographs were recorded 2 hours after the enzymatic treatment ended.

### DETAILED DESCRIPTION OF THE INVENTION

The present invention, in some embodiments thereof, relates to compounds and compositions comprising zein, and methods for zein protein functionalization, and uses the same for e.g., nutritional applications.

According to one aspect, there is provided a compound comprising a zein protein, and a polypeptide from a protein extract, linked (also referred to as “cross-linked”) to each other. In some embodiments, the zein protein and the polypeptide of a protein extract are linked to each other via a covalent bond. In some embodiments, the covalent bond comprises at least one tyrosine moiety. In some embodiments, the covalent bond comprises at least one tyrosine moiety bonded to a moiety selected from lysine, tyrosine and cysteine.

In some embodiments, the zein protein and the polypeptide of the protein extract are linked via a physical bond. In some embodiments, the physical bond is derived from, without being limited thereto, aromatic interactions, hydrophobic interactions, hydrogen bonding, or electrostatic interactions.

Herein throughout, the term “extract”, or any grammatical derivative thereof includes but is not limited to products (e.g., tinctures, concretes, absolutes, oils, essential oils, oleoresins, terpenes, terpene-free fractions, distillates, glycolic extracts, lipo-soluble extracts, dry powder extracts, fluid extracts and residues) obtained from a source (such as a plant or animal) through an extraction process such as distillation, organic extraction, alcoholic extraction, aqueous extraction and solvent extraction.

Compositions

According to one aspect, there is provided a composition comprising a plurality of the compounds disclosed herein. In some embodiments, the plurality of the compounds is in the form selected from, without limitation, an agglomerate of particles, a particulate matter, a plurality of particles, aggregates, or clusters. In some embodiments, the composition is in the form of oil-in-water emulsion. In some embodiments, the composition is in the form of a semi-solid matter, e.g., a gel or a paste form.

In some embodiments, the particles of the composition described herein are characterized by size distribution in the range of from 10 to 2000 micrometers, from 10 to 500 micrometers, from 10 to 350 micrometers, from 100 to 2000 micrometers, from 200 to 1500 micrometers or from 500 to 1000 micrometers. In some embodiments, at least 25% of the particles are characterized by a size of at least 10 micrometers. In some embodiments, at least 25% of the particles are characterized by a size of at least 15 micrometers. In some embodiments, at least 25% of the particles are characterized by a size of at least 20 micrometers. In some embodiments, at least 25% of the particles are characterized by a size of at least 50 micrometers. In some embodiments, at least 25% of the particles are characterized by a size of at least 100 micrometers. In some embodiments, at least 25% of the particles are characterized by a size of at least 200 micrometers. In some embodiments, at least 25% of the particles are characterized by a size of at least 400 micrometers. In some embodiments, at least 25% of the particles are characterized by a size of at least 600 micrometers. In some embodiments, at least 25% of the particles are characterized by a size of at least 800 micrometers. In some embodiments, at least 25% of the particles are characterized by a size of at least 1000 micrometers.

In some embodiments, the composition is characterized by a storage modulus (G′) of at least 200 Pa. In some embodiments, the composition is characterized by a storage modulus of at least 400 Pa. In some embodiments, the composition is characterized by a storage modulus of at least 800 Pa. In some embodiments, the composition is characterized by a storage modulus of at least 1000 Pa. In some embodiments, the composition is characterized by a storage modulus of at least 1500 Pa. In some embodiments, the composition is characterized by a storage modulus of at least 2000 Pa. In some embodiments, the composition is characterized by a storage modulus of at least 2500 Pa. In some embodiments, the composition is characterized by a storage modulus in the range of from 200 to 1000, from 400 to 800, from 1000 to 3000, from 2000 to 3000 or from 200 to 400 Pa.

In some embodiments, the composition is characterized by a storage modulus (G′) higher than loss modulus (G″). In some embodiments, the composition is characterized by a storage modulus at least 0.4 order of magnitude higher than G″. In some embodiments, the composition is characterized by a storage modulus at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9 or at least 2 orders of magnitude higher than G″, including any value and range therebetween.

In some embodiments, the composition is characterized by a storage modulus (G′) higher than the correspondent non-crosslinked composition. In some embodiments, the composition is characterized by a storage modulus at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.5, at least 3, at least 3.5, at least 4, at least 4.5, or at least 5 orders of magnitude higher than the correspondent non-crosslinked composition, including any value and range therebetween.

In some embodiments, the phrase “storage modulus” refers to a measure of elastic response of a material. In some embodiments, the phrase “loss modulus” refers to a measure of the viscous property of a fluid. In some embodiments, the term “zein protein” refers to any fraction containing zein protein. In some embodiments, the zein protein is sourced from a corn plant, corn cultivar, or a corn processing waste. In some embodiments, the zein protein is selected from: alpha-zein, beta-zein, gamma-zein and delta-zein.

In some embodiments, the term “zein” as used herein refer to native zein and modified zein. In some embodiments, the term “modified zein” refers to zein proteins having an amino acid sequence which is not normally occurring, which behave similarly to native zeins, and which are soluble in alcohol. Amino acid substitutions, especially those which do not substantially modify the hydrophobicity, may be introduced. For example, amino acid substitution within the repeated sections, single amino acid substitution, as well as substitutions in the segments connecting the domains of repeated sequences may be employed. Also, insertions and substitutions can be made in both the COOH— terminus and the NH2 terminus of the zein molecule.

As used herein, the terms “peptide”, “polypeptide” and “protein” are used interchangeably to refer to a polymer of amino acid residues. The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

In some embodiments, the polypeptide of the protein extract described herein, is selected from, without being limited thereto, an animal protein, a plant protein, or an algae protein. In some embodiments, the polypeptide of the protein extract described herein is selected from: a purified protein, an isolated protein fraction, a protein hydrolysate, or any combination thereof.

In some embodiments, the protein extract is a plant protein. In some embodiments, the plant protein is extracted from, without being limited thereto, potato, pea, soy, chickpea, quinoa, wheat, lentils or bean. In some embodiments, the plant protein is extracted from a potato. In some embodiments, the plant protein is extracted from a pea. In some embodiments, the plant protein is extracted from a chickpea.

In some embodiments, the polypeptide of a protein extract is an animal protein. In some embodiments, the protein is extracted from, without being limited thereto, a mammal, a bird or an insect. In some embodiments, the protein is selected from an egg protein or a whey protein.

Emulsions

According to another aspect, there is provided a composition comprising an oil-in-water emulsion, the composition comprising a zein protein and a polypeptide of a protein extract. In some embodiments, the zein protein and the polypeptide of a protein extract in the oil-in-water emulsion are linked to each other. In some embodiments, the zein protein and the polypeptide of a protein extract in the oil-in-water emulsion, are linked to each other via a covalent bond. In some embodiments, the covalent bond comprises at least one tyrosine moiety. In some embodiments, covalent bond comprises at least one tyrosine moiety covalently bonded to a moiety selected from, without being limited thereto, lysine, tyrosine and cysteine.

In some embodiments, the composition further comprises an enzyme. In some embodiments, the enzyme is a crosslinking enzyme, as described herein.

In some embodiments, the term “enzyme” as used herein refers to a “catalytically functional biomolecule,” which includes both whole native (or native-size) molecules and derivatives (e.g. genetic modifications) thereof.

In some embodiments, the term “oil” as used herein refers to any nonpolar chemical substance that is a viscous liquid at ambient temperatures and is both hydrophobic and lipophilic. In some embodiments, oil refers to lipids, fats, or any mixture thereof, either pure or containing compounds in solution. Oils can also contain particles in suspension. In some embodiments, oil is an organic oil. In some embodiments, oil is a vegetable oil. In some embodiments, oil is a plant-based oil.

In some embodiments, the term “oil-in-water emulsion” as used herein refers to a mixture of two immiscible phases wherein an oil (dispersed phase) is dispersed in an aqueous solution (the continuous phase).

In some embodiments, the composition comprises zein protein in a weight content in the range of from 0.1% to 10%. In some embodiments, the zein protein weight content is in the range of from 1% to 7%. In some embodiments, the zein protein weight content is in the range of from 1.5% to 4%. In some embodiments, the zein protein weight content is about 1%, 2%, 3%, 4%, 5%, 6%, 7%, 8%, 9%, or 10%, including any value and range therebetween. In some embodiments, the zein protein weight content is about 0.3%, 0.4%, 0.5%, 1%, 1.5%, 2%, 2.5%, 3%, 3.5%, 4%, 4.5%, or 5%, including any value and range therebetween. In some embodiments, the zein protein weight content is 1%, 1.2%, 1.4%, 1.6%, 1.8%, 2%, 2.2%, 2.4%, 2.6%, 2.8%, or 3%, including any value and range therebetween.

In some embodiments, the weight content of the polypeptide of the protein extract, in the composition described herein, is in the range of from 1% to 25%. In some embodiments, the weight content of polypeptide of a protein extract, in the composition described herein, is in the range of from 4% to 15%. In some embodiments, the weight content of polypeptide of a protein extract is in the range of from 5% to 10%. In some embodiments, the weight content of polypeptide of a protein extract is 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, including any value and range therebetween. In some embodiments, the weight content of the polypeptide of the protein extract is 3%, 3.5%, 4%, 4.5%, 5%, 5.5%, 6%, 6.5%, 7%, 7.5%, or 8%, including any value and range therebetween. In some embodiments, the weight content of the polypeptide of the protein extract is 5%, 5.2%, 5.3%, 5.4%, 5.6%, 5.8%, 6%, 6.2%, 6.4%, 6.6%, 6.8%, or 7%, including any value and range therebetween.

In some embodiments, the oil in the oil-in-water emulsion, is an edible oil. In some embodiments, the oil is of a plant source or an animal source. In some embodiments, the oil is selected from, without being limited thereto, a vegetable oil or a fruit oil. In some embodiments, the oil is selected from, without being limited thereto, a corn, cottonseed, olive, palm, sunflower, sesame, peanut or a soybean, avocado, canola, coconut flaxseed, or a pumpkin oil. In some embodiments, the oil is an olive oil. In some embodiments, the oil is a corn oil. In some embodiments, the animal sourced oil is selected from fish oil or an animal fat.

In some embodiments, the oil weight content in the oil-in-water emulsion is in the range of from 1% to 80%. In some embodiments, the oil weight content in the oil-in-water emulsion is in the range of from 10% to 50%. In some embodiments, the oil weight content in the oil-in-water emulsion is in the range of from 25% to 50%. In some embodiments, the oil weight content in the oil-in-water emulsion is in the range of from 35% to 45%. In some embodiments, the oil weight content in the oil-in-water emulsion is in the range of from 45% to 80%. In some embodiments, the oil weight content is 1%, 2%, 3%, 4%, 5%, 6%, 7%, 8%, 9%, or 10%, including any value and range therebetween. In some embodiments, the oil weight content is 10%, 15%, 20%, 25%, 30%, 35%, 40%, 45%, or 50%, including any value and range therebetween. In some embodiments, the oil weight content is 20%, 22%, 24%, 26%, 28%, 30%, 32%, 34%, 36%, 38%, 40%, 42%, 44%, 46%, 48%, or 50%, including any value and range therebetween. In some embodiments, the oil weight content is 35%, 36%, 37%, 38%, 39%, 40%, 41%, 42%, 43%, 44%, or 45%, including any value and range therebetween.

In some embodiments, the zein protein and the polypeptide of a protein extract are linked to each other by a cross-linking enzymatic reaction.

In some embodiments, the cross-linking enzyme described herein may be utilized in several levels selected from, without being limited thereto, a whole cell producing the enzyme, a cell extract containing the enzyme, an isolated enzyme or a purified enzyme.

In some embodiments, the enzyme is characterized by an enzymatic activity in temperature range of from 20 to 65° C. In some embodiments, the enzyme is characterized by an enzymatic activity in temperature range of from 15° C. to 80° C., from 25° C. to 50° C., or from 30° C. to 40° C. In some embodiments, the enzyme is characterized by an enzymatic activity in temperature of 20° C., 25° C., 30° C., 35° C., 40° C., 45° C., 50° C., 55° C., 60° C., 65° C., 70° C., 75° C., or 80° C., including any value and range therebetween. In some embodiments, the enzyme is characterized by an enzymatic activity in temperature of 25° C., 26° C., 27° C., 28° C., 29° C., 30° C., 31° C., 32° C., 33° C., 34° C., 35° C., 36° C., 37° C., 38° C., 39° C., or 40° C., including any value and range therebetween. In some embodiments, the enzyme is characterized by an enzymatic activity in temperature of 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C., including any value and range therebetween. In some embodiments, the enzyme is characterized by an enzymatic activity in temperature of about 37° C.

In some embodiments, the enzyme is characterized by an enzymatic activity in the pH range of from 5 to 11.5. In some embodiments, the enzyme is characterized by an enzymatic activity in the pH range of from 3 to 12, 5 to 9, or 6 to 8. In some embodiments, the pH conditions for the cross-linking enzymatic reaction is 3, 4, 5, 6, 7, 8, 9, 10, 11, or 12, including any value and range therebetween. In some embodiments, the enzyme is characterized by an enzymatic activity in the pH value of 5, 5.5, 6, 6.5, 7, 7.5, 8, 8.5, or 9, including any value and range therebetween. In some embodiments, the enzyme is characterized by an enzymatic activity in the pH value of 6, 6.2, 6.4, 6.6, 6.8, 7, 7.2, 7.4, 7.6, 7.8, or 8, including any value and range therebetween. In some embodiments, the enzyme is characterized by an enzymatic activity in the pH value of 7. In some embodiments, the enzyme is characterized by an enzymatic activity in the pH value of 10.

In some embodiments, the enzyme is characterized by an enzymatic activity in the ionic strength range of from 10 mM to 1000 mM. In some embodiments, the enzyme is characterized by an enzymatic activity in the ionic strength range of from 10 mM to 500 mM, or 10 mM to 200 mM. In some embodiments, the enzyme is characterized by an enzymatic activity in the ionic strength of 10 mM, 50 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1000 mM, including any value and range therebetween. In some embodiments, the enzyme is characterized by an enzymatic activity in the ionic strength of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM, including any value and range therebetween. In some embodiments, the enzyme is characterized by an enzymatic activity in the ionic strength of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM, including any value and range therebetween. In some embodiments, the enzyme is characterized by an enzymatic activity in the ionic strength of 25 mM. In some embodiments, the enzyme is characterized by an enzymatic activity in the ionic strength of 50 mM.

In some embodiments, the time required for the cross-linking enzymatic reaction is in the range of from 0.1 hr to 24 hr. In some embodiments, the time required for the cross-linking enzymatic reaction is in the range of from 0.2 hr to 15 hr. In some embodiments, the time required for the cross-linking enzymatic reaction is in the range of from 0.3 hr to 10 hr. In some embodiments, the time required for the cross-linking enzymatic reaction is in the range of from 0.5 hr to 4 hr. In some embodiments, the cross-linking enzymatic reaction is obtained within lhr, 2 hr, 3 hr, 4 hr, 5 hr, 6 hr, 7 hr, 8 hr, 9 hr, 10 hr, 11 hr, 12 hr, 13 hr, 14 hr, 15 hr, 16 hr, 17 hr, 18 hr, 19 hr, 20 hr, 21 hr, 22 hr, 23 hr, or 24 hr, including any value and range therebetween. In some embodiments, the cross-linking enzymatic reaction is obtained within 0.1 hr, 0.2 hr, 0.3 hr, 0.4 hr, 0.5 hr, 0.6 hr, 0.7 hr, 0.8 hr, 0.9 hr, or lhr, including any value and range therebetween. In some embodiments, the cross-linking enzymatic is obtained within 0.5 hr, 1 hr, 1.5 hr, 2 hr, 2.5 hr, 3 hr, 3.5 hr, or 4 hr, including any value and range therebetween.

The phrase “enzyme activity” refers to moles of substrate converted per unit time. Enzyme activity is a measure of the quantity of active enzyme present and is thus dependent on conditions, which should be specified.

In some embodiments, the term “cross-linking” as used herein refers to the formation of a chemical bond between two molecules. In some embodiments, cross-linking is inter cross-linking.

In some embodiments, the cross-linking enzyme is selected from, without being limited thereto, tyrosinase, peroxidase, transglutaminase, lipoxygenase, protein sulfide reductase, protein disulfide isomerase, sulfhydryl oxidase, hexose oxidase, lysyl oxidase, amine oxidase, glucose oxidase, hexose oxidase, pentose oxidase, or laccase. In some embodiments, the cross-linking enzyme is tyrosinase.

In some embodiments, the term “tyrosinase” refers to a copper-containing enzyme that catalyzes the formation of quinones from phenols and polyphenols.

In some embodiments, the tyrosinase biological source is selected from, without being limited thereto, Bacillus megaterium, Agaricus bisporus, Trichoderma reseei, Botryosphaeria obtuse, Verrucomicrobium spinosum or a squid ink. In some embodiments, the tyrosinase biological source is Bacillus megaterium.

In some embodiments, the weight content of the cross-linking enzyme in the reaction mixture is in the range of from 0.001% to 0.5%. In some embodiments, the cross-linking enzyme weight content is in the range of from 0.01% to 0.1%. In some embodiments, the cross-linking enzyme weight content is in the range of from 0.02% to 0.05%. In some embodiments, the cross-linking enzyme weight content is 0.001%, 0.005%, 0.01%, 0.02%, 0.04%, 0.06%, 0.08%, 0.1%, 0.12%, 0.14%, 0.16%, 0.18%, 0.2%, 0.25%, 0.3%, 0.35%, 0.4%, 0.45%, or 0.5%, including any value and range therebetween. In some embodiments, the cross-linking enzyme weight content is 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, or 0.1%, including any value and range therebetween. In some embodiments, the cross-linking enzyme weight content is 0.02%, 0.022%, 0.024%, 0.026%, 0.028%, 0.03%, 0.032%, 0.034%, 0.036%, 0.038%, 0.04%, 0.042%, 0.044%, 0.046%, 0.048%, 0.05%, 0.052%, 0.054%, 0.056%, 0.058%, or 0.06%, including any value and range therebetween. In some embodiments, the cross-linking enzyme weight content is 0.033%. In some embodiments, the cross-linking enzyme weight content is 0.04%.

In some embodiments, weight content is weight per weight. In some embodiments, weight content is weight per volume.

In some embodiments, the composition described herein further comprises an enzymatic cross-linking mediator. In some embodiments, the cross-linking enzymatic reaction process further comprises the introduction of an enzymatic cross-linking mediator. In some embodiments, the enzymatic cross-linking mediator is an aromatic compound. In some embodiments, the enzymatic mediator is selected from, without being limited thereto, caffeic acid, vanillic acid, phenolic compound, dopa, tyrosine, or chlorogenic acid.

In some embodiments, the composition described herein further comprises a nutraceutical compound. In some embodiments, the nutraceutical compound is entrapped in the composition interior. In some embodiments, entrapment is driven by hydrophobic interactions between zein protein and the nutraceutical compound. In some embodiments, the nutraceutical compound is selected from, without being limited thereto, vitamins and antioxidants. In some embodiments, the nutraceutical compound is vitamin D, curcumin or lycopene.

In some embodiments, the compound and composition described herein are used for nutritional supplementation. In some embodiments, compound and composition described herein are used as alternative protein source. Non-limiting exemplary applications/uses of the disclosed composition under this section are selected from a cheese replacement, meat and sausage replacement, fish mimics, shrimp mimics, caviar mimics, mousse, cream, spread, dressing or a sauce.

Process

According to some embodiments of the present invention, there is provided a process for covalently linking a zein protein and a polypeptide of a protein extract. According to some embodiments of the present invention, there is provided a process for providing a nutritional supplement. In some embodiments, the process comprises the steps of (a) mixing a solution comprising a zein protein, a solution comprising a polypeptide of a protein extract, and a solution comprising tyrosinase thereby creating a mixture, and (b) incubating the mixture for a specified time and temperature, thereby covalently linking said zein protein and said polypeptide of said protein extract.

In some embodiments, the process comprises the steps of adding a solution comprising tyrosinase to a solution comprising a polypeptide of a protein extract hereby creating a mixture, adding a solution comprising a zein to said mixture and incubating the mixture for a specified time and temperature, thereby covalently linking said zein protein and said polypeptide of said protein extract.

In some embodiments, the process comprises the steps of (a) contacting a solution comprising a zein protein and a solution comprising a polypeptide of a protein extract, thereby making a mixture thereof, and (b) adding tyrosinase to the mixture, and (c) incubating the mixture for a specified time and temperature, thereby covalently linking said zein protein and said polypeptide of said protein extract.

In some embodiments, mixing comprises homogenizing said mixture. In some embodiments, the process comprises homogenization step. In some embodiments, homogenization is prior to incubation.

In some embodiments, a solution comprises zein protein. In some embodiments zein protein weight content is in the range of from 0.2% to 20%. In some embodiments, zein protein weight content is in the range of from 2% to 14%. In some embodiments, zein protein weight content is in the range of from 3% to 8%. In some embodiments, zein protein weight content is 2%, 3%, 4%, 5%, 6%, 7%, 8%, 9%, 10%, 11%, 12%, 13%, 14%, 15%, 16%, 17%, 18%, 19%, or 20%, including any value and range therebetween. In some embodiments, zein protein weight content is 0.2%, 0.3%, 0.4%, 0.5%, 0.6%, 0.8%, 1%, 3%, 4%, 5%, 6%, 7%, 8%, 9%, or 10%, including any value and range therebetween. In some embodiments, zein protein weight content is 2%, 2.4%, 2.8%, 3.2%, 3.6%, 4%, 4.4%, 4.8%, 5.2%, 5.6%, or 6%, including any value and range therebetween.

In some embodiments, a solution comprising a zein protein further comprises ethanol/oil mixture. In some embodiments, the ethanol weight content is in the range of from 0.2% to 50%. In some embodiments, the ethanol weight content is in the range of from 1% to 50%. In some embodiments, the ethanol weight content is in the range of from 5% to 20%. In some embodiments, the ethanol weight content is in the range of from 6% to 14%. In some embodiments, the ethanol weight content is 0.2%, 0.3%, 0.4%, 0.5%, 0.6%, 0.8%, 1%, 5%, 10%, 15%, 20%, 25%, 30%, 35%, 40%, 45%, or 50%, including any value and range therebetween. In some embodiments, the ethanol weight content is 4%, 6%, 8%, 10%, 12%, 14%, 16%, 18%, or 20%, including any value and range therebetween. In some embodiments, the ethanol weight content is 6%, 7%, 8%, 9%, 10%, 11%, 12%, 13% or 14%, including any value and range therebetween.

In some embodiments, the oil weight content is in the range of from 1% to 80%. In some embodiments, the oil weight content is in the range of from 10% to 50%. In some embodiments, the oil weight content is in the range of from 25% to 50%. In some embodiments, the oil weight content is in the range of from 35% to 45%. In some embodiments, the oil weight content is in the range of from 45% to 80%. In some embodiments, the oil weight content is 1%, 2%, 3%, 4%, 5%, 6%, 7%, 8%, 9%, or 10%, including any value and range therebetween. In some embodiments, the oil weight content is 10%, 15%, 20%, 25%, 30%, 35%, 40%, 45%, or 50%, including any value and range therebetween. In some embodiments, the oil weight content is 20%, 22%, 24%, 26%, 28%, 30%, 32%, 34%, 36%, 38%, 40%, 42%, 44%, 46%, 48%, or 50%, including any value and range therebetween. In some embodiments, the oil weight content is 35%, 36%, 37%, 38%, 39%, 40%, 41%, 42%, 43%, 44%, or 45%, including any value and range therebetween.

In some embodiments, a solution further comprises a nutraceutical compound.

In some embodiments, a solution comprises a polypeptide of a protein extract. In some embodiments, the polypeptide of a protein extract weight content is in the range of from 2% to 40%. In some embodiments, the polypeptide of a protein extract weight content is in the range of from 6% to 40%. In some embodiments, zein protein weight content is in the range of from 8% to 30%. In some embodiments, polypeptide of the protein extract weight content is in the range of from 10% to 15%. In some embodiments, polypeptide of the protein extract weight content is 2%, 3%, 4%, 5%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, or 50%, including any value and range therebetween. In some embodiments, polypeptide of the protein extract weight content is 2%, 3%, 4%, 5%, 6%, 7%, 8%, 9%, or 10%, including any value and range therebetween. In some embodiments, polypeptide of the protein extract weight content is 6%, 7%, 8%, 9%, 10%, 11%, 12%, 13%, 14%, 15%, or 16%, including any value and range therebetween. In some embodiments, polypeptide of the protein extract weight content is 10%, 10.4%, 10.6%, 10.8%, 11.2%, 11.6%, 12%, 12.4%, 12.8%, 13.2%, 13.6%, or 14%, including any value and range therebetween.

In some embodiments, one or more proteins are in their native state.

As used herein the term “native” refers to 1) conditions that do not disrupt intermolecular interactions within peptides or proteins that allow them to maintain a three dimensional structure that is either a three dimensional structure of the protein as found in nature or synthesized in a cell-free in vitro translation system, or 2) to proteins having a three dimensional structure that is the same or substantially the same as a three dimensional structure of the protein as found in nature or synthesized in a cell-free in vitro translation system. A three-dimensional structure can be a secondary, tertiary, or quaternary structure of a protein. In some embodiments, the term “native” refers to nondenaturing or nondenatured.

In some embodiments, a solution comprises a polypeptide of a protein extract is an aqueous solution.

In some embodiments, the process further comprises adding a cross-linking enzyme to the mixture. In some embodiments, the cross-linking enzyme weight content in the mixture is in the range of from 0.001% to 0.5%. In some embodiments, the cross-linking enzyme weight content is in the range of from 0.01% to 0.1%. In some embodiments, the cross-linking enzyme weight content is in the range of from 0.02% to 0.05%. In some embodiments, the cross-linking enzyme weight content is 0.001%, 0.005%, 0.01%, 0.02%, 0.04%, 0.06%, 0.08%, 0.1%, 0.12%, 0.14%, 0.16%, 0.18%, 0.2%, 0.25%, 0.3%, 0.35%, 0.4%, 0.45%, or 0.5%, including any value and range therebetween. In some embodiments, the cross-linking enzyme weight content is 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, or 0.1%, including any value and range therebetween. In some embodiments, the cross-linking enzyme weight content is 0.02%, 0.022%, 0.024%, 0.026%, 0.028%, 0.03%, 0.032%, 0.034%, 0.036%, 0.038%, 0.04%, 0.042%, 0.044%, 0.046%, 0.048%, 0.05%, 0.052%, 0.054%, 0.056%, 0.058%, or 0.06%, including any value and range therebetween. In some embodiments, the cross-linking enzyme weight content is 0.033%.

In some embodiments, the process further comprises incubating the mixture at the conditions described herein (e.g. time, temperature, pH, and ionic strength as described above).

In some embodiments, the mixture is incubated with shaking. In some embodiments, the shaking is in the range of 100 to 300 rpm. In some embodiments, the shaking is in the range of 100 to 200 rpm. In some embodiments, the shaking is in the range of 200 to 300 rpm. In some embodiments the shaking is 100 rpm, 150 rpm, 200 rpm, 210 rpm, 220 rpm, 230 rpm, 240 rpm, 250 rpm, 260 rpm, 270 rpm, 280 rpm, 290 rpm, or 300 rpm, including any value and range therebetween.

In some embodiments, an emulsion was obtained by homogenizing the mixture using a shear dispersing unit. In some embodiments, a shear dispersing unit is selected from a low-speed-shear dispersing unit, a high-speed-shear dispersing unit, a friction dispersing unit, a high-pressure-jet dispersing unit, an ultrasonic dispersing unit. In some embodiments, an emulsion was obtained by homogenizing using a Pro200, Pro-Scientific.

In some embodiments, a shear dispersing unit is used at a speed ranging from 1000 rpm to 40000 rpm. In some embodiments, a shear dispersing unit is used at a speed ranging from 20000 rpm to 40000 rpm. In some embodiments, a shear dispersing unit is used at a speed ranging from 30000 rpm to 40000 rpm. In some embodiments, a shear dispersing unit is used at a speed of 30000 rpm, 31000 rpm, 32000 rpm, 33000 rpm, 34000 rpm, 35000 rpm, 36000 rpm, 37000 rpm, 38000 rpm, 39000 rpm, or 40000 rpm, including any value and range therebetween.

In some embodiments, the dispersion time is in the range of 0.1 min to 10 min. In some embodiments, the dispersion time is in the range of 1 min to 10 min. In some embodiments, the dispersion time is in the range of 0.1 min to 5 min. In some embodiments, the dispersion time is in the range of 1 min to 5 min. In some embodiments, the dispersion time is 0.1 min, 0.5 min, 1 min, 2 min, 3 min, 4 min, 5 min, 6 min, 7 min, 8 min, 9 min, or 10 min, including any value and range therebetween.

In some embodiments, the dispersion temperature is in the range of 0° C. to 150° C. In some embodiments, the dispersion temperature is in the range of 20° C. to 50° C. In some embodiments, the dispersion temperature is in the range of 20° C. to 40° C. In some embodiments, the dispersion temperature is in the range of 30° C. to 40° C.

In some embodiments an emulsion is obtained by high-pressure homogenization. In some embodiments, high-pressure homogenization is performed in a high-pressure homogenization device. In some embodiments, high-pressure homogenization is performed in a EmulsiFlex™-C3. In some embodiments high-pressure homogenization is done in 1 passes, 2 passes, 3 passes, 4 passes, 5 passes, 6 passes, 7 passes, 8 passes, 9 passes, or 10 passes, including any value and range therebetween.

In some embodiments the pressure used for high-pressure homogenization is in the range of 1 kPsi to 50 kPsi. In some embodiments the pressure used for high-pressure homogenization is in the range of 100 psi to 50 kPsi. In some embodiments the pressure used for high-pressure homogenization is in the range of 10 kPsi to 40 kPsi. In some embodiments the pressure used for high-pressure homogenization is 1 kPsi, 5 kPsi, 10 kPsi, 20 kPsi, 30 kPsi, 40 kPsi, or 50 kPsi, including any value and range therebetween. In some embodiments the pressure used for high-pressure homogenization is 20 kPsi.

In some embodiments, an emulsion has a stability of more than 3 days. In some embodiments, an emulsion has a stability of more than 5 days. In some embodiments, an emulsion has a stability of more than 7 days. In some embodiments, an emulsion has a stability of more than 12 days. In some embodiments, an emulsion has a stability of more than 15 days. In some embodiments, an emulsion has a stability of more than 20 days. In some embodiments, an emulsion has a stability of more than 30 days.

In some embodiments, the process further comprises adding an enzymatic cross-linking mediator to the mixture.

In some embodiments, the process further comprises adding a nutraceutical compound to the mixture.

According to one aspect, there is provided a compound comprising a polypeptide derived from zein protein, and from another molecule having a moiety selected from primary amine, phenol and thiol, or any combination thereof. In some embodiments, the polypeptide and the molecule are linked to each other via a covalent bond. In some embodiments, the covalent bond comprises at least one phenolic group. In some embodiments, the phenolic group is covalently bonded to the other molecule via a moiety selected from a primary amine, phenol and thiol.

In some embodiments, the molecule described herein is an organic molecule. In some embodiments, the organic molecule is a natural sourced or a synthetic molecule.

In some embodiments, the molecule is a polymer. In some embodiments, the molecule is a biopolymer. In some embodiments, the biopolymer is selected from, without being limited thereto, proteins, polysaccharides and polynucleic acids.

In some embodiments, the molecule is a therapeutic agent. In some embodiments, the therapeutic agent is an edible drug or a food supplement. In some embodiments, the therapeutic agent is selected from, without being limited thereto, antibiotics, antiparasitic agents, antioxidants and vitamins.

In some embodiments, the composition comprises the zein protein and the molecule described herein, is in the form of oil-in-water solution.

In some embodiments, the zein protein and the molecule described herein are linked to each other by a cross-linking tyrosinase enzymatic process.

General

As used herein the term “about” refers to ±10%.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. Any particular embodiment of the invention may include a plurality of “optional” features unless such features conflict.

As used herein and in the appended claims, the singular forms “a,” “and,” and “the” include plural referents unless the context clearly dictates otherwise.

Also, the use of “or” means “and/or” unless stated otherwise. Similarly, “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of”

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### EXAMPLES

Reference is now made to the following examples, which together with the above descriptions illustrate some embodiments of the invention in a non-limiting fashion.

Materials and Methods

Materials.

Nile red was obtained from Sigma Chemical Co. (Rehovot, Israel); sodium chloride from BioLab Ltd. (Jerusalem, Israel), 2-propanol and ethanol from Gadot-Group (Netanya, Israel). Corn oil and was purchased from the local supermarket (Haifa, Israel), CGM was provided by Galam Ltd, Israel, while the pea protein concentrate (PPC) was purchased from Raab Vitalfood GmbH (Rohrbach, Germany).

Enzymatic crosslinking of pea protein concentrate and zein.

PPC was suspended in 50 mM Tris-HCl pH=10 and stirred overnight at 4° C. Dispersion of PPC was centrifuged at 7500 rpm for 5 min and the supernatant with soluble protein (concentration of 0.5 and 1% w/w) was used. Zein (0.5% w/v) was suspended in 50 mM Tris-HCl pH=10 and stirred overnight at 4° C. The crosslinking reaction was carried out with soluble PPC (concentration of 1% w/w), or a mixture of PPC (0.5% w/w) and zein (0.5% w/w). TyrBm was added in 1:25 enzyme to protein ratio, while the non-crosslinked samples were treated similarly but without enzyme addition. The reaction mixtures were incubated at 37° C. with shaking at 250 rpm in an incubator shaker (TU-400 Orbital Shaker Incubator, MRC, Holon) for 4 h. Samples were taken at various time points (0, 30, 60, 120, 180 and 240 min, respectively) and the reaction was stopped by directly mixing the reaction mixture with electrophoresis sample buffer (×4) at 1:1 ratio (v/v). The samples were analyzed by SDS-PAGE.

Preparation of crosslinked protein solutions and emulsions.

Non-crosslinked and TyrBm-crosslinked emulsions with PPC alone and PPC-zein were fabricated. 8% (w/v) PPC was suspended in 50 mM Tris-HCl pH=10 and stirred overnight at 4° C. Dispersion of PPC was centrifuged at 7500 rpm for 5 min to remove high size protein aggregates and the supernatant with a protein concentration of 2% (w/w) was further used. The reaction conditions were set as described in previous section comprising solutions of 60% PPC-buffer and 40% (v/v) corn oil. A coarse emulsion was obtained when the mixture was homogenized using a shear dispersing unit (Pro200, Pro-Scientific) for 1 min at 35000 rpm. A fine emulsion was obtained by high-pressure homogenization (EmulsiFlex-C3, Avestin Inc., Ottawa, ON, Canada) for 3 passes at 20 kPsi. The emulsions were then incubated for 120 min at 37° C. with shaking at 100 rpm. For emulsions with zein addition, 2% (w/v) zein was dissolved in ethanol/corn oil mixture. The reaction conditions and fabrications steps remain the same as described above comprising solutions of 60% PPC-buffer and 40% (v/v) zein in corn oil. For the non-crosslinked emulsions, all details remain similar while buffer was added instead of enzyme.

SDS-PAGE analysis.

SDS-PAGE was performed on a discontinuous buffered system using 15% separating gel and 4% stacking gel. The samples were heated for 10 min at 95° C., after addition of sample buffer (4×), 1:1 (v/v). Samples were mixed with reducing (5% β-mercaptoethanol) sample buffer. The gels were stained with 0.25% Coomassie brilliant blue (R-250) in 50% ethanol and 10% acetic acid, and destained in 10% acetic acid [methanol:acetic acid: water, 20:10:70 (v/v/v)]

Emulsion Characterization.

Emulsions were analyzed using a variety of techniques including visual observation, determination of composition and percentage of adsorbed proteins, particle size analysis, rheological measurements and inverted microscopy.

Determination of composition and percentage of adsorbed proteins (AP).

Each emulsion was centrifuged at 10,000×g for 30 min at ambient temperature resulting with two phases: the creamed oil droplets at the top of the tube and the aqueous phase of the emulsion at the bottom. The subnatant was extracted using a syringe and then filtered through a 0.22 μm filter (Millipore Corp.). The protein concentration of the filtrate (C_{f}) was determined with the Bradford method. The starting protein solution was also centrifuged at the same conditions to determine the protein concentration (Cₛ) in the supernatant. The AP % was calculated as follows:

AP %=(Cs−Cf)×100/Cs (1)

For the evaluation of the composition of crosslinked protein in the bulk and the interface during the reaction, 1 ml of the coarse and fine emulsions, and emulsions setting for 2 h of incubation were evaluated. Emulsions were centrifuged (10,000×g for 30 min/RT) and the cream layer was washed and re-dispersed in 50 mM Tris-HCl pH=10 and the resultant re-dispersion was then centrifuged at the same condition. The adsorbed proteins in the cream layer were directly extracted with an equal volume of an electrophoresis sample buffer, namely 0.25 M Tris-HCl buffer (pH 6.8) containing 4% (w/v) SDS, 20% (w/v) glycerol and 2% 2-mercaptoethanol. After stirring for more than 20 h, the mixtures were centrifuged at 10,000×g for 30 min to obtain the subnatants (containing adsorbed proteins) for SDS-PAGE experiments. The two phases were analyzed by SDS-PAGE.

Particle size distribution (PSD).

Particle size distribution of the emulsions was measured by laser diffraction particle size analyzer MasterSizer 3000 (Malvern Instruments Ltd, Malvern, Worcestershire, UK) with a wet sample dispersion unit (Malvern Hydro MV, UK). The optical properties were defined as refractive index 1.47 (corn oil) and 1.330 (dispersant water) and absorption index 0.001. Emulsion samples were diluted with distilled water (1:1 w/w) with or without addition of SDS (1% w/w).

Rheological measurements at small deformation.

Emulsions were transferred to the rheometer (DHR-2, TA Instruments, DE, USA) fitted with the parallel plates geometry (d=40 mm) with the gap of 1 mm. The temperature was kept constant at 25° C. For apparent viscosity measurements, shear rate was increased from 0.5 to 100 s⁻¹ as a function of shear stress and viscosity was recorded. Oscillatory frequency sweep (0.01-10.0 Hz) measurements were performed at 1% strain within the LVR region (determined previously). The equipment was controlled using the Trios program (TA Instruments, DE, USA).

Direct observation of emulsions.

Visualization of the emulsion microstructure was evaluated with a Cell Observer inverted microscope (Zeiss Axiovert 200, Jena, Germany) using Nile red to label oil phase of emulsion. Nile red (0.1 mg mL⁻¹ in ethanol) was mixed with the emulsions in ratio 1:50 followed by 10 min incubation in ice before being placed on a glass microscope slide and covered. Images were processed by the AxioVision (Zeiss) image analysis software for acquisition and image processing.

Experimental design and analysis.

All experiments were conducted in triplicate, and results were expressed as mean±standard deviation. Statistical analyses were performed using Microsoft Excel 2013 data analysis tool pack. The paired sample t-tests assuming equal variances with a level of significance of 95% was applied to compare means.

### Example 1: Method of Enzymatic Crosslinking of Potato Proteins and Zein

1% (w/v) potato protein (PP) was suspended in 25 mM phosphate buffered saline (PBS) pH=7 and stirred for 30 min 4° C. ambient temperature. The crosslinking reaction was carried while enzyme to protein ratio was lowered to 1:30. As for zein, 1% (w/v) zein was suspended in 50 mM SPB pH=10 and stirred overnight at 4° C. The crosslinking reaction was then carried out. Samples were taken at various time points (0, 30, 60, 120, 180 and 240 min, respectively) and the reaction was stopped by directly mixing reaction mixture with electrophoresis sample buffer (×4) at 1:1 ratio (v/v). The samples were analyzed by SDS-PAGE.

### Example 2: Preparation of Crosslinked Protein Solutions and Emulsions

6.1% (w/v) PP was suspended in 25 mM SPB pH=7 and stirred overnight at 4° C. temperature to ensure full hydration. 2% (w/v) of zein was dissolved in ethanol in olive oil mixer overnight at 4° C. temperature. The reaction conditions were set as described above and immediately 40% (w/v) olive oil was added and homogenized using a shear dispersing unit (Pro200, Pro-Scientific) for 5 min at 35000 rpm. The emulsions were then incubated for 60 or 240 min at 37° C. with shaking at 250 rpm in an incubator shaker (TU-400 Orbital Shaker Incubator, MRC, Holon, Israel). For the non-crosslinked emulsions, all details remain similar while SPB was added instead of enzyme.

### Example 3: TYRBM-Catalyzed Crosslinking of Zein and Potato Protein

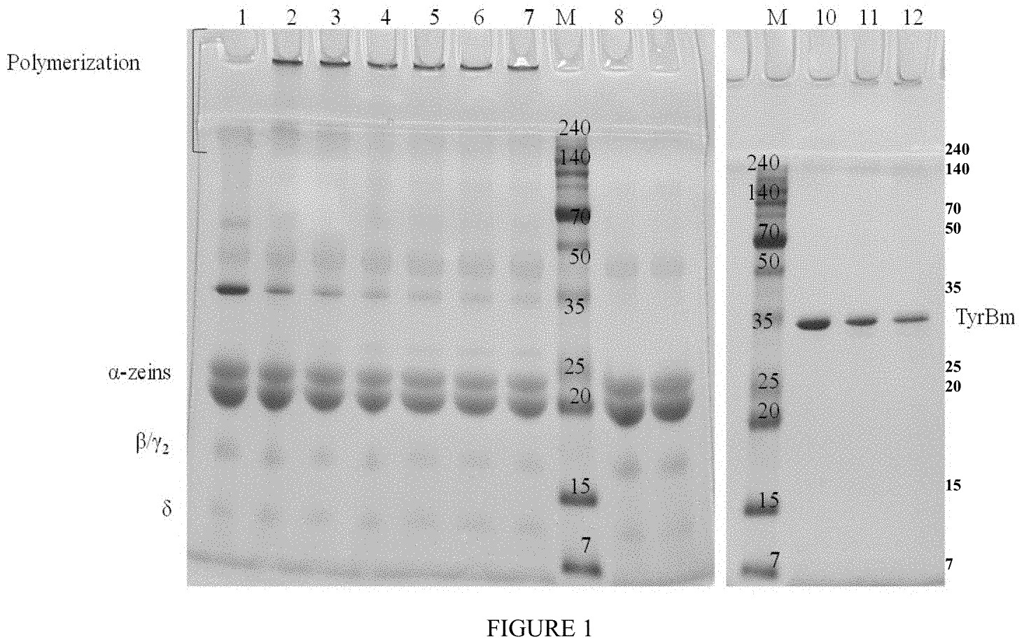

Before applying tyrosinase on the complex emulsion system, its effect on native zein was evaluated. Zein dispersion in buffer was incubated with tyrosinase derived from Bacillus megaterium (TyrBm) for different time periods and crosslinking was evaluated using SDS-PAGE (FIG. 1). The formation of high-molecular weight fragments was indeed visualized with the reduction in the intensity of the protein monomer bands even after 30 minutes of incubation (FIG. 1). α-zeins seem to be good substrates for TyrBm, followed by β/γ₂ and δ zein fractions. Self-crosslinking of TyrBm was also evident from the control reaction (no zein).

### Example 4: Characterization of Pp-Zein Stabilized Emulsions

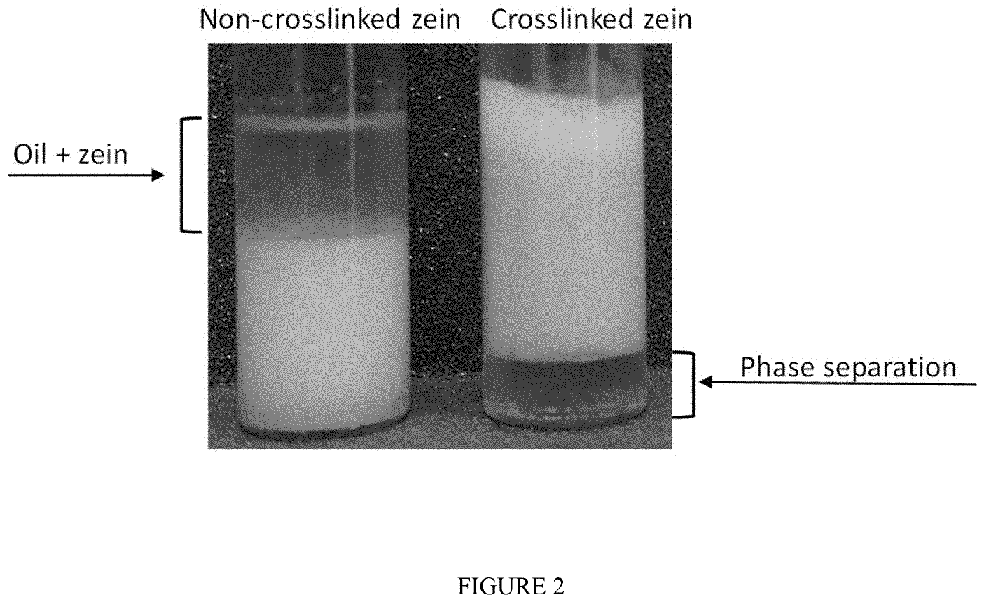

The influence of polymerization degree on emulsion properties was evaluated when zein stabilized emulsions were incubated for 4 hours with TyrBm (or buffer, for the non-crosslinked system). Both the crosslinked and non-crosslinked zein emulsions were unstable and separation was visible immediately after incubation (FIG. 2), thus this system was not used in further experiments. Subsequently, the potato protein rich fraction dispersed in an aqueous phase in combination with zein dispersed in the oil phase was used for fabrication of concentrated o/w emulsions.

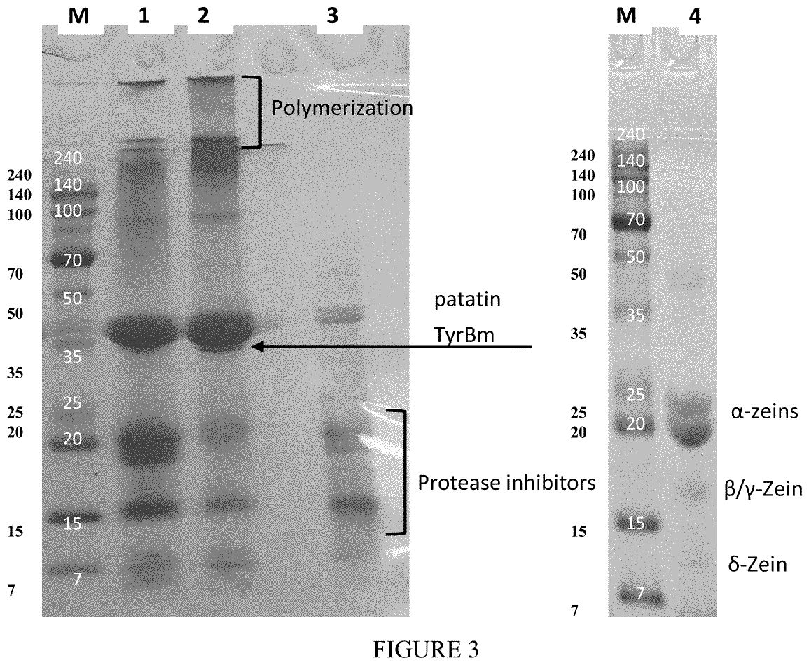

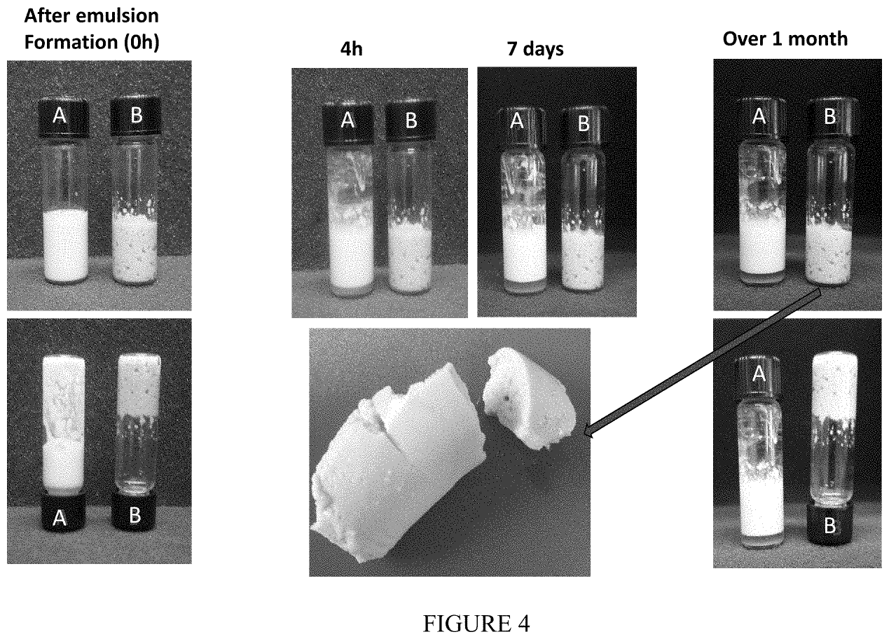

Successful TyrBm crosslinking of mainly potato protease inhibitors and α-zeins was evident by the decrease in their band intensities and formation of high molecular weight bands as visualized in the SDS-PAGE gel (FIG. 3). In contrast to the non-crosslinked emulsion, which separated a few hours after incubation, the TyrBm-crosslinked emulsion was stable even after 1 month of storage (FIG. 4). TyrBm crosslinking induced the formation of a self-standing aerated gel-like structure after 4 hours of incubation, while the non-crosslinked emulsion remained liquid (FIG. 4). It is assumed that TyrBm crosslinked potato protease inhibitors together with α-zeins contributed to the formation of the gelled structure. Despite of the presence of zein, which is known as a gelling and thickening agent, an unstable emulsion was obtained in the absence of tyrosinase.

Particle Size Distribution

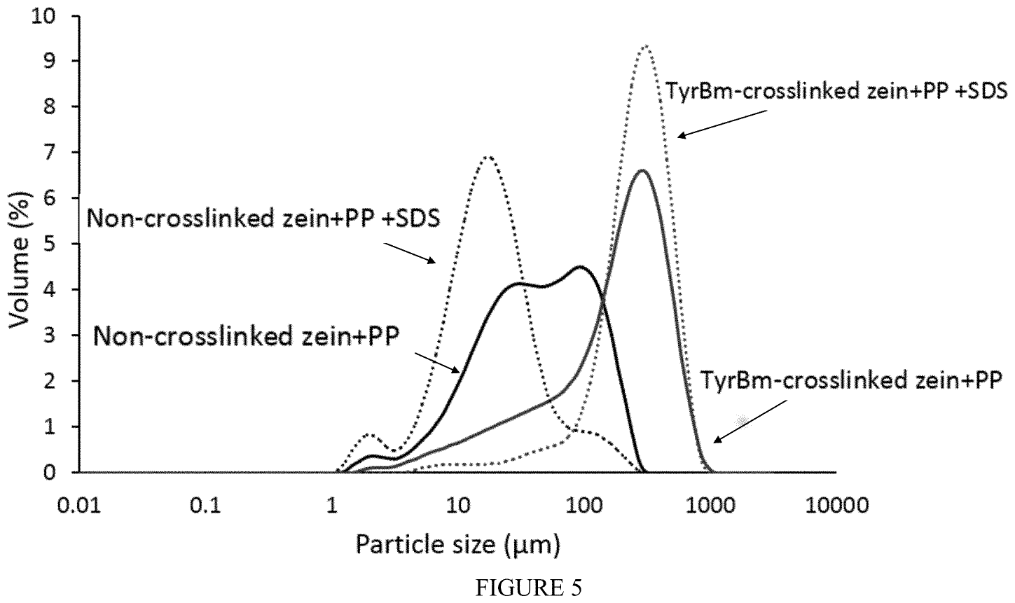

The non-crosslinked oil-in-water emulsions obtained using the PP rich fraction and zein showed a polydispersity with a trimodal droplet size distribution (FIG. 5). The major population had the mean diameter of 98.7 μm, while the smallest peak was attributed to the minor fraction of oil droplets (2.13 μm). In the presence of 1% SDS, the major population of non-crosslinked zein-PP stabilized emulsions decreased in size to a mean diameter 18.7 μm suggesting the formation of flocculation in oil droplets, mainly through hydrophobic interactions. In contrast, the TyrBm-crosslinked zein-PP stabilized emulsion had a uniform monomodal particle size distribution with a mean diameter of droplets of 310 μm (FIG. 5), suggesting extensive covalent polymerization of crosslinked zein-PP complex. In the presence of 1% SDS little changes could be observed in the droplet size (distribution), eliminating possibility of hydrophobic interactions between oil droplets and confirming formation of covalent bonds by TyrBm action.

Accelerated Physical Stability of Emulsions

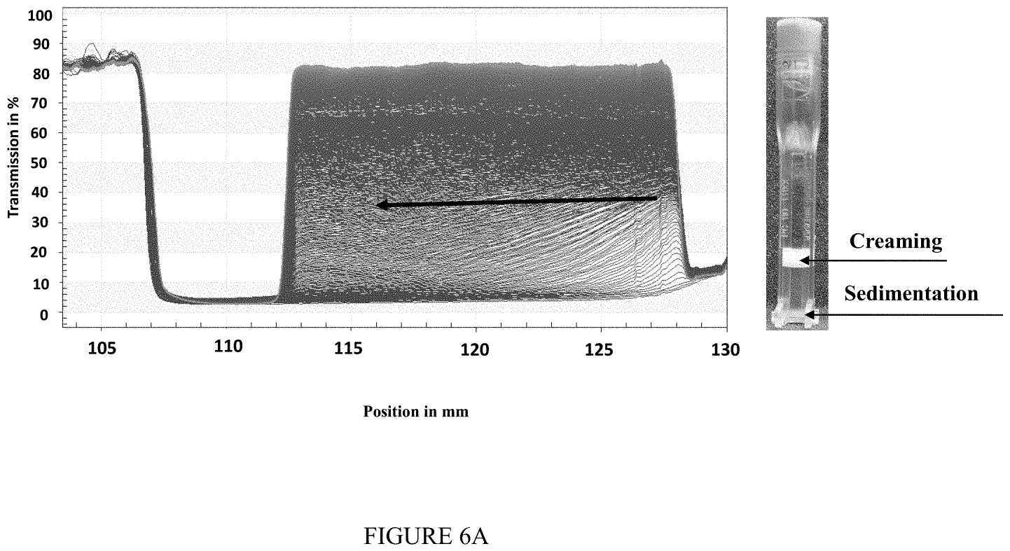

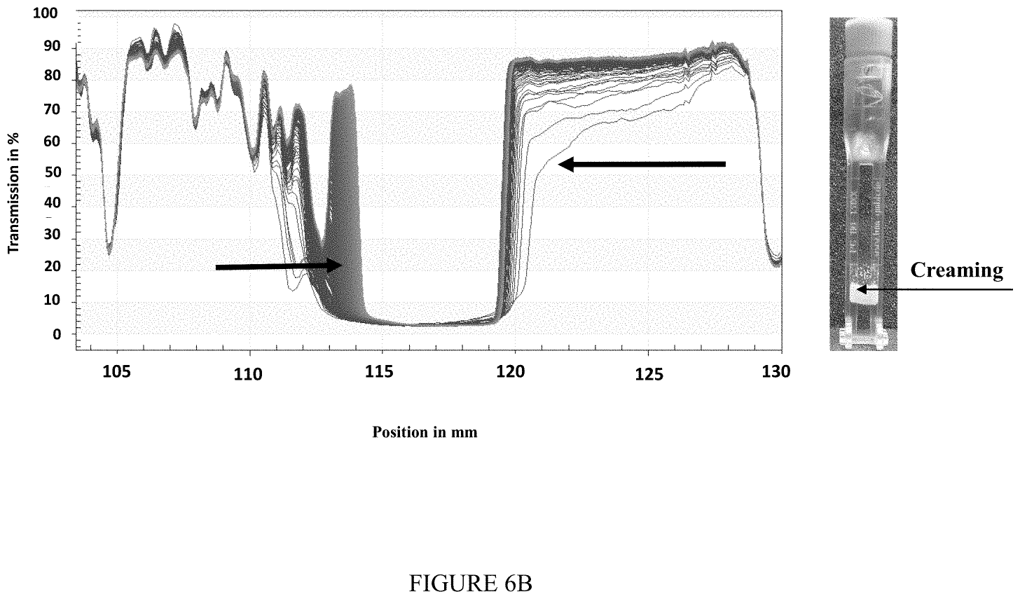

Analytical centrifugation and the time and space-resolved transmission extinction profiles are generally used when creaming/sedimentation velocity of an emulsion is calculated. This method is not appropriate for gel-like emulsions due to different properties of the gel matrix. Nonetheless, instability phenomena of creaming and sedimentation were determined for both emulsions. Both measures of instability were observed for the non-crosslinked emulsions, while crosslinking by TyrBm eliminated sedimentation (FIGS. 6A-B). On the other hand, instability (creaming or sedimentation) was not affected by tyrosinase crosslinking of PP-stabilized emulsions when zein was not present. Thus, it is presumed that α-zeins and potato protease inhibitors were crosslinked by TyrBm and they formed an elastic network, which contributed to elimination of sedimentation.

Rheological Properties of Emulsions

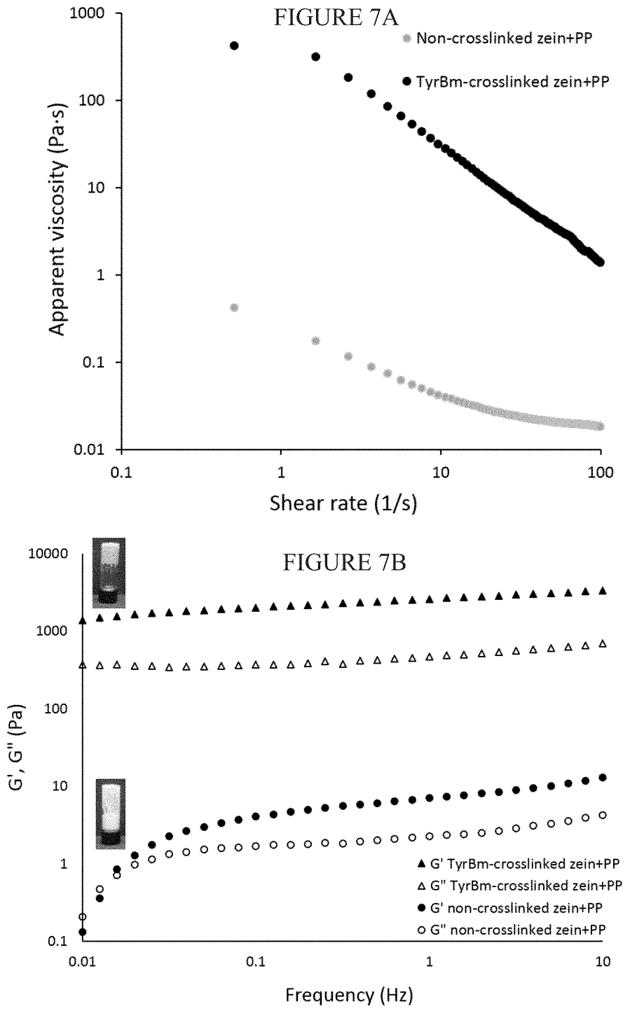

The relationship between apparent viscosity and shear rate of the non-crosslinked and crosslinked emulsion are shown in FIGS. 7A-B. Rheological behaviour was found to be related to emulsion stability. TyrBm-crosslinked emulsion exhibited pseudoplastic behavior; i.e., the curve displayed shear-thinning behavior in the range of shear rate up to 100 1/s, while the non-crosslinked emulsion showed also shear-thinning behavior, but less pronounced. The shear-thinning behavior was previously reported for food emulsions due to a dramatic shear-induced structural breakdown.

Shear-thinning behavior was observed for PP-stabilized o/w emulsion in combination with a thickening agent such as guar gum or chitosan, as well as in zein-stabilized glycerol emulgel. Viscosity of TyrBm crosslinked emulsions measured at 200 1/s was over 40-fold higher than the non-crosslinked sample (Table 1). Thus, the oil droplets obtained more dissipation energy associated with fluid flow, resulting in the higher viscosity of the emulsion. When the shear rate increased, the hydrodynamic forces dominated and disrupted the flocs, causing a reduction in the viscosity in the entire emulsion system. Complete deformation and disruption of oil droplets was observed when the viscosity of the emulsions reached a constant value. The texture modification of non-crosslinked and TyrBm-crosslinked emulsion was further evaluated using the frequency sweep test.

TABLE 1
Rheological properties of non-crosslinked and TyrBm-crosslinked PP and zein
stabilized o/w emulsion: shear viscosity (at 200 s⁻¹), storage modulus
(G′), loss modulus (G″) (at 1 Hz) and tan δ after 4 hours of incubation.

| | G′ (Pa) | G″ (Pa) | η_{200 1/s} (Pa · s) | Tan δ |
|---|---|---|---|---|
| Non-crosslinked | 7.02 ± 1.97^{a} | 2.25 ± 0.63^{a} | 0.014 ± 0.006^{a} | 0.323 ± 0.165^{a} |
| Crosslinked | 2580 ± 24.82^{b} | 466.03 ± 0.81^{b} | 0.611 ± 0.993^{b} | 0.197 ± 0.031^{b} |

Different letters indicate significant differences at p < 0.05 according to t- test. Comparison was made between non-crosslinked and crosslinked samples for each parameter.
Values are the average of three experiments.

In FIG. 7 the higher storage modulus (G′) vs. loss modulus (G″) is evident for both emulsions, indicating formation of predominantly elastic gel-like nature. The crosslinked emulsion showed mostly frequency independent behavior, while the non-crosslinked emulsion was frequency dependent, which is characteristic of weak gels. For an ideal gel which behaves elastically, the G′ is expected to be independent of frequency and G′>>G″. According to this, a strong gel structure was formed by TyrBm crosslinking of PP and zein and the storage modulus was considerably higher than the loss modulus. Furthermore, the crosslinked emulsion had 2.5-orders of magnitude higher G′ values compared to the non-crosslinked emulsion. At the frequency of 1 Hz, more than 300-fold higher G′ value was reported for TyrBm-crosslinked emulsion in comparison to non-crosslinked emulsion, while damping factor (tan δ) was 1.6-fold higher, respectively (Table 1). Tan δ takes into account the contribution of both elastic and viscous modulus (tan δ=G″/G′), and provides information on the balance of the viscoelastic moduli of a material. The lower the tan δ, the higher the contribution of the elastic component of these materials, and as can be expected, a stronger elastic component would contribute to increased structural heterogeneity, i.e. reducing the miscibility of the material with the saliva and making it harder to swallow. The higher values of tan δ for the non-crosslinked emulsion, coupled with low values of storage modulus throughout the mechanical spectrum would suggest a less stiff gel structure with greater contribution of the viscous component.

A higher storage modulus indicates the presence of more covalent bonds. The absence of the gel-like emulsion formation without applying TyrBm, confirmed that TyrBm is essential, despite previously reported results for zein as crucial in forming emulsion gels. Without being bound by any particular mechanism, this suggests that TyrBm played a positive role of intensifying the emulsion elastic property leading to a better-structured network of aerated emulsion gel. Therefore, coalescence and flocculation were prevented due to stronger mutual acting force of oil droplets.

Textural Properties of Emulsions

Texture profile analysis (TPA) is an important imitative test for textural characterization, performed as a two-bite compression test, and it provides a link between mechanical properties and textural attributes during oral processing. The textural properties of non-crosslinked and TyrBm-crosslinked PP-zein stabilized o/w emulsions are shown in Table 2. The crosslinked emulsion had a significantly firmer texture (2-fold higher hardness) than non-crosslinked emulsion (p<0.05). Hardness is used to estimate the maximum force required to compress the sample during the first bite and is related to the strength of the gel network. Gumminess is an indicator of the amount of work needed to make a food sample ready to swallow, while the cohesiveness measures the structural strength of internal bonds, which holds the food matrix together in a bolus and prevents it from disintegrating into fragments during swallowing. Enzymatic treatment led to increase in gumminess and resilience (p<0.05), while cohesiveness was slightly lower compared to non-crosslinked emulsion. The crosslinked emulsion's higher elasticity is also characterized by a 9-fold increase in resilience compared with the non-crosslinked emulsion. The rheological measurements in small deformation strengthen these results with the higher G′ values and viscosity for the TyrBm-crosslinked emulsion.

TABLE 2
TPA parameters of non-crosslinked and TyrBm-crosslinked
PP and zein stabilized o/w emulsion.

| Parameter | Non-crosslinked | TyrBm-crosslinked |
|---|---|---|
| Hardness (N) | 0.23 ± 0.01^{a} | 0.44 ± 0.09^{b} |
| Cohesiveness (—) | 0.61 ± 0.03^{a} | 0.56 ± 0.02^{a} |
| Parameter | Non-crosslinked | TyrBm-crosslinked |
| Gumminess (N) | 0.14 ± 0.001^{a} | 0.21 ± 0.03^{b} |
| Resilience (—) | 0.03 ± 0.01^{a} | 0.26 ± 0.15^{b} |

Different letters indicate significant differences at p < 0.05 according to t- test.
Comparison was made between non-crosslinked and crosslinked sample for each parameter.
Values are the average of three experiments

Microstructure of Emulsions

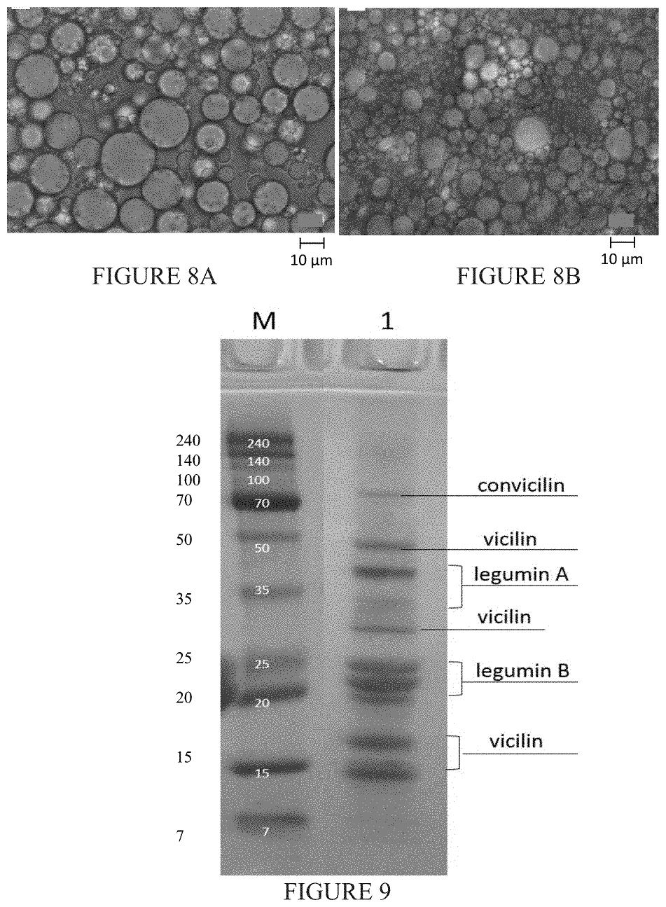

Finally, the microstructure of TyrBm-crosslinked or non-crosslinked PP-zein stabilized emulsions were characterized with a Cell Observer inverted microscope, as shown in FIG. 8. The micrograph of the non-crosslinked emulsion showed structure typical for concentrated emulsions (FIG. 8A). The TyrBm-crosslinked emulsion exhibited tighter network with non-spherical droplets (FIG. 8B). The close packing of oil droplets is most likely the consequence of covalent protein crosslinks formed by TyrBm, which contributed to formation of a gel-like structure. Despite the fact that greater particle size values were detected in the PSD analysis (FIG. 5), a tighter structure with seemingly smaller particles was observed in the microscope. As seen in the micrographs all oil droplets were present in a closely associated form with a maximum droplet size of 20 μm (FIG. 8B). The obtained results resemble images corresponding to strong gel-like emulsions, however air bubbles or formation of large aggregates could not be observed in the micrographs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### Example 5: Pea Protein and Zein Characterization

Crosslinked pea protein concentrate (PPC) was used for fabrication of emulsions. Characterization of commercial PPC was performed using SDS-PAGE (FIG. 9), presenting typical pea protein pattern. It has been reported that pea seeds contain between 20-30% wt of total protein composed of 7S/11S globulin (50-60% of total) and albumin 2S (15-25%). Hexameric homo-oligomer, legumin (11S) and trimeric vicilin/convicilin (7S) are the main globulin protein species. Legumin (˜360-400 kDa) has each subunit (˜60 kDa) made up of acidic (38-40 kDa) and basic polypeptides (19-22 kDa) linked by a single disulfide bond. Legumin subunit has approximately four methionine residues and between 2-7 cysteine amino acid residues. Vicilin 7S is a trimeric protein with molecular weight ˜150 kDa that lacks sulfur amino acids and hence cannot form disulfide bonds. Pea vicilin subunits composition varies mostly because of post-translation processing, thus it might consist of ˜47 kDa, ˜50 kDa, ˜34 kDa and ˜30 kDa subunits. Furthermore, some of the 50-kDa subunits of vicilin are nicked by posttranslational proteolysis soon after biosynthesis, and the resulting peptides (ranging from 12.5 to 33 kDa) remain associated with the intact subunits in the native 150-kDa oligomer. Convicilin (˜290 kDa), a third storage protein, has a subunit of ˜71 kDa associated in trimers or tetramers. Pea protein fractions of convicilin (˜70 kDa), legumin A (40 kDa), legumin B (20 kDa) and vicilin (50 kDa, 30-35 kDa, below 20 kDa) can be clearly observed on SDS-PAGE gel (FIG. 9). Proteomic analysis confirmed pea protein identity (data not shown) and the protein content was 81% according to elemental analysis. Based on solubility tests, pH 10 was chosen for all experiments. As previously mentioned, zein solubility is limited to alkaline pH and aqueous-alcohols, thus zein was dispersed in an ethanol/oil solution. All four fractions of zein proteins, extracted from CGM, were clearly distinguished by SDS-PAGE analysis (data not shown), as previously reported. The most abundant fraction in CGM purified zein is α-zein (80-85%) consisting of two proteins with molecular weight of 22 and 24 kDa, followed by less abundant fractions β (17 kDa), γ (presented by two subunits γ₁˜27 kDa and γ₂˜18 kDa) and δ (10 kDa), as previously reported.

### Example 6: Tyr-Bm-Catalyzed Crosslinking of Pea Proteins and Zein-Pea Combinations

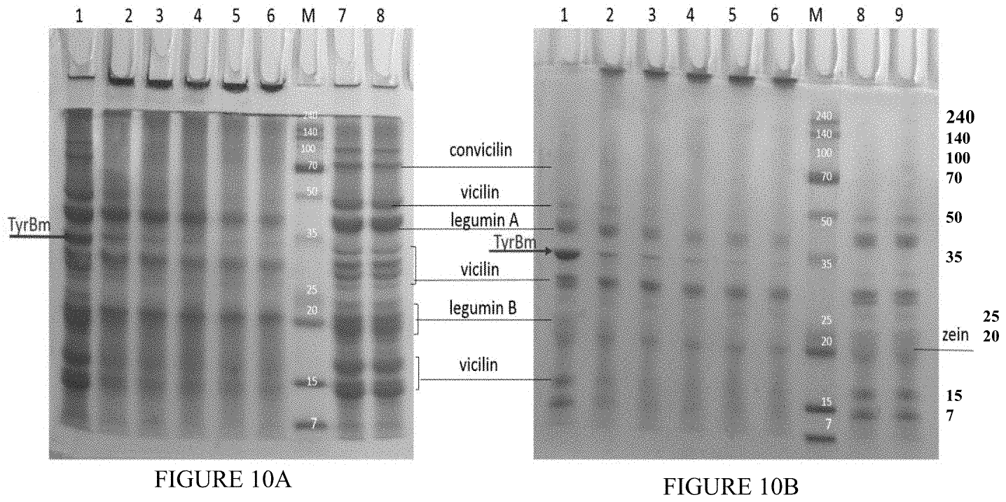

TyrBm ability to catalyze the crosslinking of pea protein alone, and zein with pea protein was evaluated. The polymer formation from pea protein fractions was rapid and concurrent formation of high molecular bands was observed within 30 minutes from the enzyme addition (FIG. 10A). A decrease in band intensity with time suggested that the main proteins to be crosslinked were vicilin/convicilin fractions and low molecular weight fractions of vicilin (below 20 kDa). Convicilin and legumin A are more abundant in tyrosine residues than vicilin (50 kDa) and legumin B (protein sequences in UniProtKB: P13915, P15838, P13918 and P14594 respectively), thus they are expected to be better substrates for tyrosinase. However, vicilin fraction with molecular weight around 32 kDa and fractions of legumin A/B were crosslinked, but to a lesser extent compared to convicilin and other vicilin fractions (50 kDa, 34 kDa, 30 kDa, below 20 kDa) that completely disappeared by the end of the incubation time (FIG. 10A). Not only the abundancy but also availability/accessibility of the target residues to the solvent/enzyme which is related to their position in the globular structure is crucial for enzymatic catalysis. The same pattern for pea protein fractions was reported when transglutaminase was used for crosslinking. According to several authors, vicilin fractions of 31/33 kDa and legumin B fractions could not be crosslinked by transglutaminase without preceding chemical or thermal treatment, while vicilin fractions with lower molecular weight (below 20 kDa) were still unaffected by the enzyme. According to our results, low molecular weight pea protein fractions were good substrates for TyrBm. In general, pea protein fractions seem to be better substrates for TyrBm, compared to potato protein fractions. Potato proteins have slightly higher amount of aromatic amino acids (phenylalanine+tyrosine) compared to the pea proteins, however it seems that tyrosine residues are more available to TyrBm active site in pea protein than in potato protein. In the case of crosslinking a mixture of pea proteins and zein by TyrBm, the disappearance of pea protein fractions vicilin and convicilin was observed, as well as a decline in α-zein fractions (FIG. 10B). Crosslinked biopolymers had molecular weight higher than 240 kDa and were not able to enter stacking gel (FIG. 10B).

### Example 7: Characterization of Emulsions

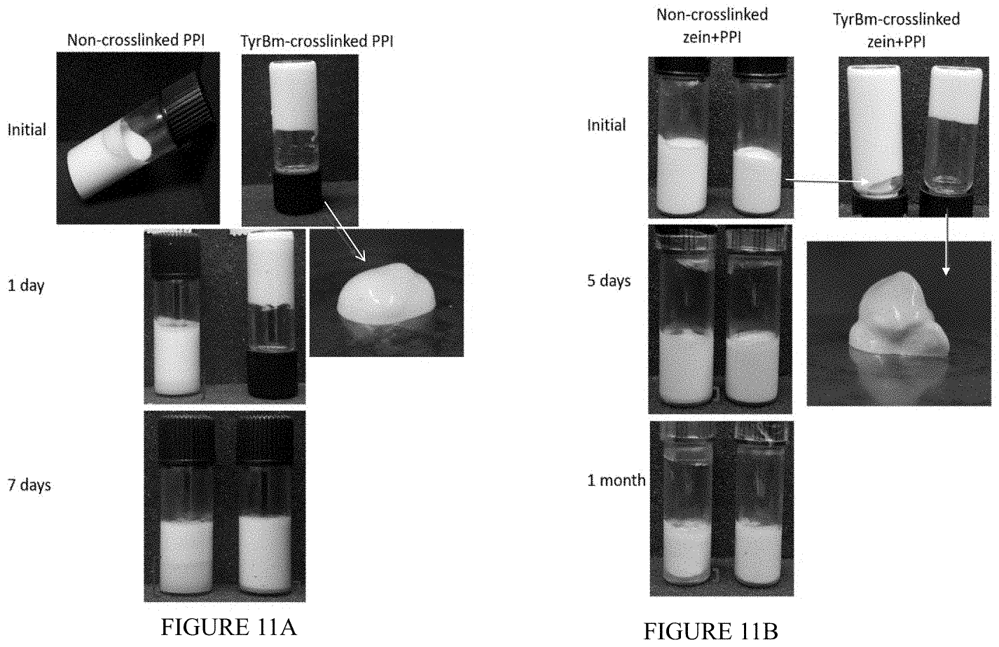

The effect of TyrBm crosslinking on emulsion properties was evaluated by comparing non-crosslinked and TyrBm-crosslinked pea protein-stabilized emulsions incubated for 2 hours. As observed on the FIG. 11A the crosslinked emulsion had paste-like structure resembling mayonnaise, while the non-crosslinked emulsion remained as a liquid. The paste-like structure contributed to the longer stability of the crosslinked emulsion (7 days), while non-crosslinked pea protein-stabilized emulsion was unstable and phase separation occurred 6 h after incubation (FIG. 11A). Similar results reporting prolonged stability and development of a gel-like structure induced by applying TyrBm, were found for potato protein concentrated (40%) o/w emulsions, and soy-glycinin o/w emulsions (10%). Furthermore, development of a gel-structure of pea protein emulsion alone or in combination with milk protein crosslinked by transglutaminase was reported recently. In addition, by using high concentrations of pea protein (6% or 10% w/v) at higher values (above 0.5) a gel-like structure was obtained without enzyme addition. The formation of aggregates by TyrBm crosslinking of pea convicilin/vicilin fractions contributed the most to the emulsion stability and development of gel-like network, as observed on SDS-PAGE (FIG. 10A).

In order to achieve better physical stability of the concentrated emulsion, zein was incorporated. The fabrication steps were similar with the pea protein and TyrBm in the aqueous phase while zein was dispersed in the oil phase. The non-crosslinked zein-pea protein emulsion exhibited prolonged stability (5 days) compared to the non-crosslinked pea protein alone stabilized emulsions (6 h) (FIGS. 11A-B), emphasizing the importance of zein to the emulsion stability. Exceeding the non-crosslinked zein-pea protein emulsion, characterized by liquid behavior and phase separation after 5 days of storage, the TyrBm-crosslinked zein-pea protein emulsion showed gel-like structure and stability against phase separation even after 1 month of storage at room temperature (FIG. 11B). The same results were reported for TyrBm-crosslinked zein-potato protein emulsion. Furthermore, it is assumed that TyrBm-crosslinking of pea protein, particularly convicilin and vicilin fractions along with α-zein fractions contributed to the formation of the gelled structure and more pronounced emulsion stability.

### Example 8: Composition and Percentage of Adsorbed and Non-Adsorbed Proteins

The protein adsorbed layer formed at the fluid-fluid interface protects the colloidal system against various mechanisms of destabilization. Thus, the percentage of adsorbed proteins (AP %) for any stage of emulsion production (coarse/fine emulsion, emulsion after 2 h of incubation) were examined. It was observed that more than 50% of the protein is in adsorbed state after applying high pressure homogenization (Table 3).

TABLE 3
Adsorbed proteins percentage (AP %) in the non-crosslinked and TyrBm-crosslinked
pea/and zein protein stabilized emulsion at different fabrication steps.

| | Pea protein | | Zein-pea protein | |
|---|---|---|---|---|
| | stabilized emulsion | | stabilized emulsion | |
| Processing step | Non-crosslinked | TyrBm-crosslinked | Non-crosslinked | TyrBm-crosslinked |
| Coarse emulsion¹ | 17.26 ± 3.64 | 14.55 ± 3.38 | 30.84 ± 2.59 | 28.89 ± 2.90 |
| Fine emulsion² | 60.46 ± 1.41 ^{a} | 65.07 ± 1.22^{b} | 59.01 ± 0.74 ^{a} | 63.63 ± 1.11 ^{b} |
| 1 h after incubation³ | 59.01 ± 1.63 ^{a} | 53.81 ± 1.25 ^{b} | 59.19 ± 1.55 ^{a} | 65.10 ± 1.26 ^{b} |
| 2 h after incubation³ | 57.18 ± 1.57 ^{a} | 53.24 ± 1.24 ^{b} | 56.09 ± 2.66 ^{a} | 66.91 ± 0.53 ^{b} |

Each value is the mean and standard deviations of at least duplicate measurements.
Means with different letters in the same row are significantly different at p < 0.05 according to t-test.
Comparison was made between non-crosslinked and crosslinked pea protein (or zein-pea protein) samples for each emulsion stage:
¹After shear homogenization at 35000 rpm/1 min
²After high-pressure homogenization for 3 passes at 40 kPsi.
³37° C./100 rpm shaking

After high pressure homogenization and during the incubation period, TyrBm-crosslinked emulsions had significantly higher (p<0.05) adsorbed protein percentage compared to the non-crosslinked emulsions, emphasizing the advantage of the enzymatic treatment. As previously reported, strengthening of interfacial layers and the associated enhancement of colloid stability can be achieved by increasing the degree of protein—protein crosslinking. When shear homogenization was applied, AP % was relatively low (below 30%) for all emulsions, and the difference in AP % between crosslinked and non-crosslinked emulsions was not significant. It has been reported that surface hydrophobicity of pea proteins increased upon high-pressure homogenization, due to the disruption of disulfide-bonded large aggregates into smaller ones leading to exposure of buried hydrophobic amino acids and higher values of AP %. Moreover, after centrifugation of the coarse emulsion, precipitation of small amounts of insoluble proteins was observed, while no precipitation occurred in the fine emulsions (data not shown). This suggests that during the high-pressure homogenization, insoluble proteins can be adsorbed at the interface, leading to higher AP % in the fine emulsions. Once the insoluble proteins are adsorbed, they can form much thicker interfacial film at the adsorption sites. However, this interfacial film was disrupted by stirring/shaking during incubation with/without TyrBm, which was observed as a decrease in AP % for pea protein emulsions, as well as for non-crosslinked zein-pea protein emulsions. In contrast, an increase in AP % was observed for TyrBm-crosslinked zein-pea protein emulsion. The adsorption rate of protein molecules at the oil-water interface is mainly determined by molecular size and structure of proteins. The increased AP % of the TyrBm crosslinked zein-pea protein complex at the oil/water interface compared to other emulsions could be related to the higher hydrophobicity of the TyrBm-crosslinked complex, as previously reported for the complex of pea-arabic gum. In this case, hydrophobic residues of the zein-pea protein crosslinked complex could be exposed towards the oil phase, while the hydrophilic groups of pea protein remain masked inside the complex. Furthermore, a slightly lower AP % in the crosslinked pea protein-stabilized emulsion was obtained compared to the crosslinked zein-pea protein emulsion. The reason for this could be related to features of the crosslinked aggregates and differences in their structure due to zein incorporation. When acidic conditions or heat treatment were applied to pea proteins, similar results were obtained for protein adsorption at the o/w interface. In addition, when pea protein (2% w/w) was used in emulsion production (ϕ0.2) at low pH, percentage of adsorbed protein was 65%, which is similar to the present results, while at lower oil (ϕ0.1) and protein concentrations (2% w/w), AP % was around 20%.

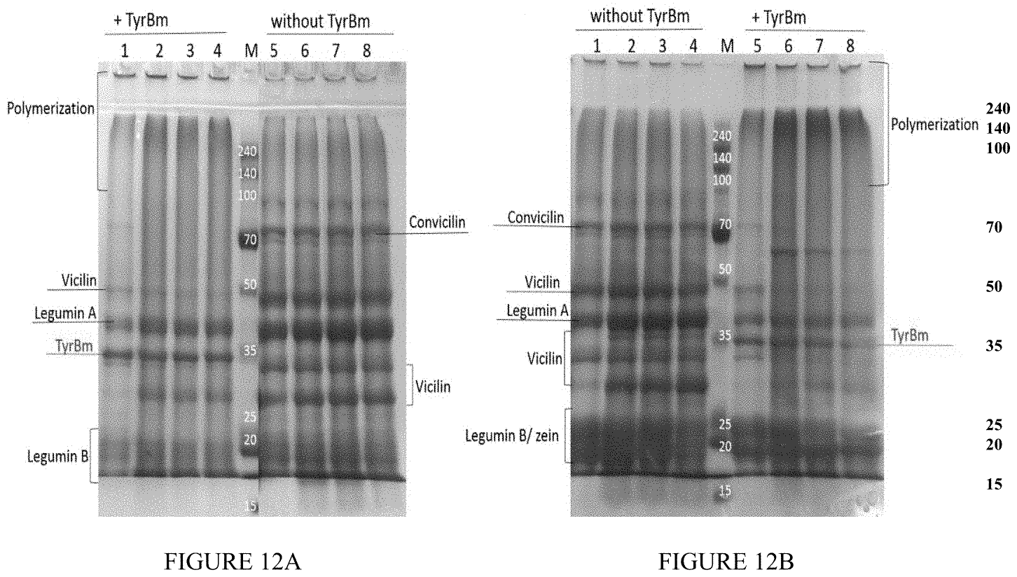

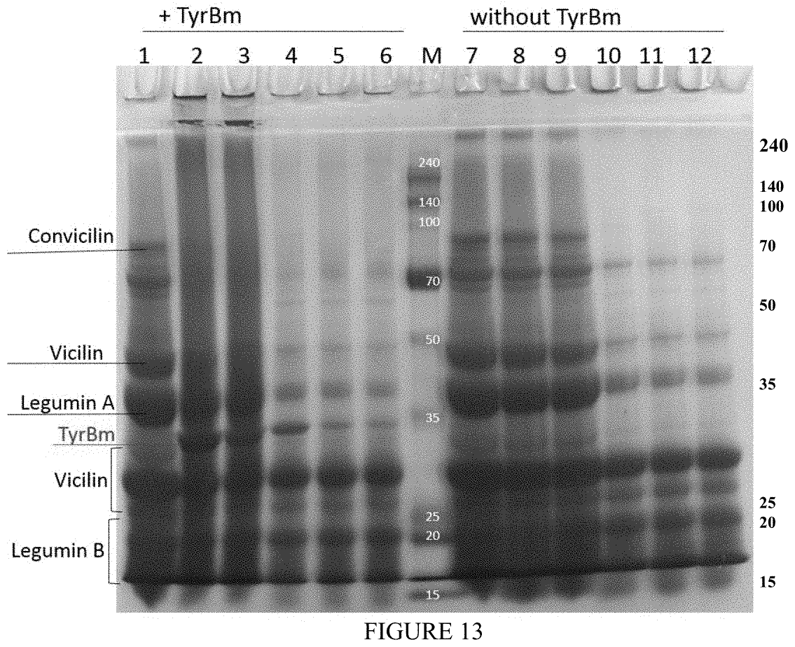

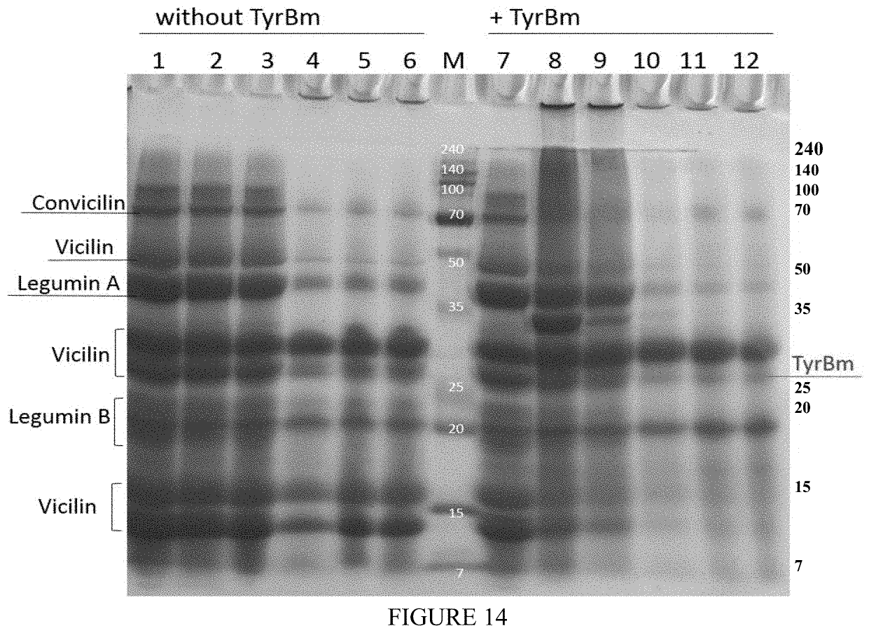

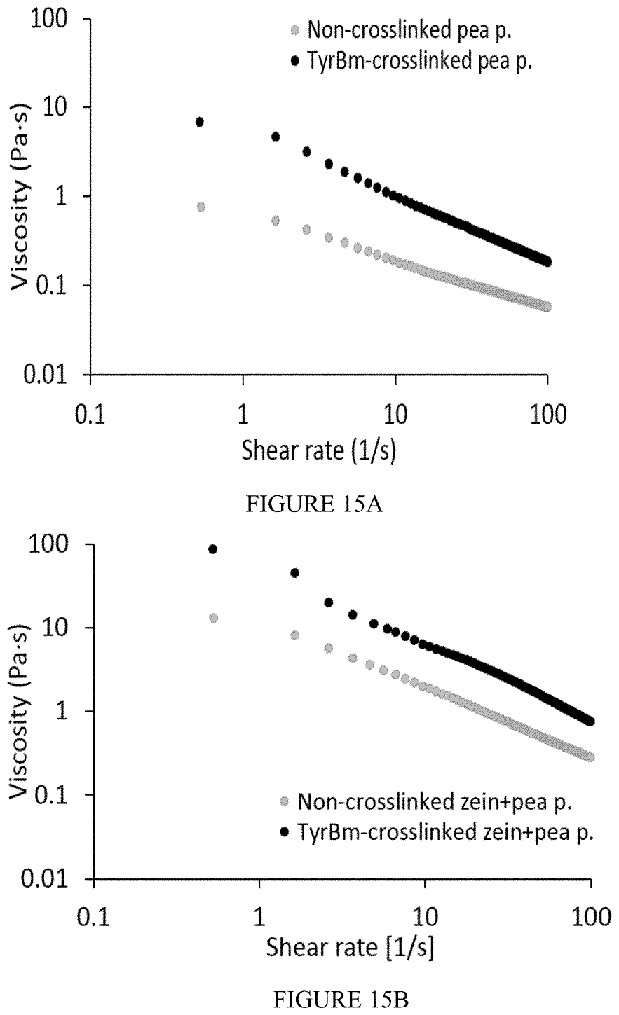

SDS-PAGE analysis under reducing conditions was conducted in order to investigate the composition of adsorbed and non-adsorbed proteins in the produced emulsions (FIGS. 12A-B, FIG. 13, FIG. 14). Non-adsorbed pea proteins mostly included vicilin fractions (34 and below 20 kDa) and legumin B (FIG. 13), as reported previously. As shown in FIG. 12A, almost all of the pea protein fractions were adsorbed at the o/w interface during high pressure homogenization. The adsorbed proteins mainly included of convicilin, legumin A and vicilin fractions (50 kDa, 30 kDa), which is in accordance with previous reports. Zein fractions were adsorbed at the interface of oil droplets, as expected due to their high hydrophobicity (FIG. 12B). According to the obtained results, the enzymatic treatment was favorable for the protein adsorption at the interface. The crosslinking reaction took place in the non-adsorbed protein in the bulk (FIG. 13, FIG. 14) as well as in the interface (FIGS. 12A-B). Moreover, after high pressure homogenization, TyrBm was adsorbed at the interface and was able to crosslink pea and zein proteins. A decline in convicilin/vicilin fractions during the incubation with TyrBm and formation of covalently cross-linked biopolymers in the stacking gel, indicates on adsorption of crosslinked pea proteins on the interface of oil droplets (FIG. 12A). The same trend was observed when zein was present in system, resulting in the formation of zein-vicilin/convicilin complexes at the interface of the oil droplets (FIG. 12B). Covalent interactions induced by TyrBm between zein and pea protein fractions at the oil surface are also responsible for the formation of a unique and strong viscoelastic protein film surrounding the oil droplets, confirmed by rheological measurements (FIGS. 15A-B). The obtained results correlated with studies on the crosslinking of glycinin-stabilized emulsions by TyrBm as well as soy protein-stabilized emulsions by transglutaminase. As observed in the SDS-PAGE gel of non-adsorbed and adsorbed proteins (FIGS. 12A-B, FIG. 13, FIG. 14) in both non-crosslinked emulsions, protein complexation was observed between pea protein fractions and zein-pea protein fractions, indicating that complexation is taking place in both the bulk and interface. The high-pressure homogenization in combination with alkaline pH can alter pea protein conformation and aggregation properties, leading to the creation of new intra- or intermolecular bonds among pea protein fractions, resulting in pea protein and pea-zein protein complexation.

### Example 9: Particle Size Distribution

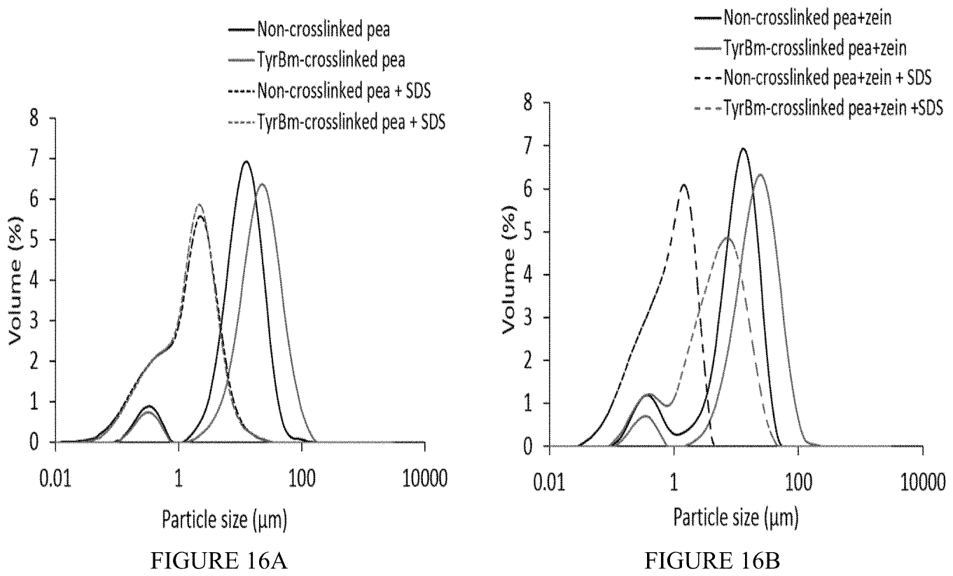

The droplet size distributions of diluted pea protein and zein-pea protein stabilized o/w emulsions with/without SDS are presented in FIG. 16. The non-crosslinked and crosslinked emulsions showed bimodal droplet size distribution with one dominant population. The crosslinked emulsions had a larger mean diameter of the dominant population compared to the non-crosslinked emulsion (FIGS. 16A-B). However, in the presence of 1% SDS, the major population of non-crosslinked and crosslinked pea protein stabilized emulsions decreased to a similar mean diameter suggesting the presence of flocculation, formed through hydrophobic interactions, as was previously reported for pea protein emulsions prepared at various pH values and heat treatment. However, crosslinking of pea protein fractions in the crosslinked emulsion were strongly confirmed by SDS-PAGE gel of non-adsorbed and adsorbed proteins (FIGS. 12A-B), as well as by a different structure and stability of the crosslinked pea protein emulsions (FIG. 11A). The lack of significant differences in mean droplet diameter between crosslinked and non-crosslinked pea protein emulsions with SDS addition could be due to crosslinking of pea protein fractions adsorbed on the o/w interfaces of the same droplet, not between different droplets. Similar results were reported when bovine serum albumin (BSA) was crosslinked by transglutaminase. Additionally, it seems that oil droplets were stabilized due to high percentage of adsorbed crosslinked pea protein complexes, while the network of oil droplets was stabilized by hydrophobic forces which were disrupted by SDS.

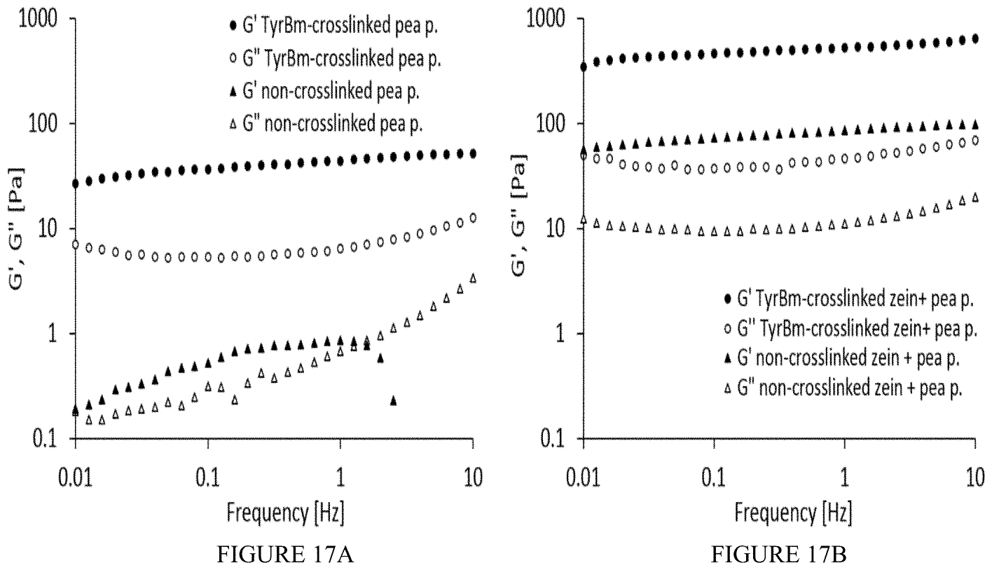

Zein incorporation followed by TyrBm action led to aggregate formation of oil droplets, which was confirmed by PSD with SDS addition (FIG. 16B), as well as by rheological measurements (FIGS. 17B-D). The mean droplet diameter of crosslinked zein-pea protein emulsions after SDS addition decreased compared to samples without SDS, while it still remained larger compared to the non-crosslinked sample with SDS addition. As mentioned for pea protein crosslinked emulsions, the driving force in emulsion stability of crosslinked zein-pea protein system was provided through the stabilization of oil droplets by adsorption of crosslinked zein-pea protein complexes as well as partially crosslinking of proteins on different droplets, while hydrophobic forces enhanced the oil droplets network. However, in the case of TyrBm crosslinked zein-potato proteins stabilized emulsions, PSD remained the same after SDS addition, which could be due to the different protein types and 3-fold higher protein concentration in the continues phase compared to the non-crosslinked zein-pea protein emulsion. Thus, the enhanced aggregation of oil droplets in the zein-potato protein system suggests that crosslinking occurred between protein fractions adsorbed on different droplets, as well as on the same droplets, as previously shown for β-lactoglobulin crosslinked with transglutaminase.

### Example 10: Rheological Properties of Emulsions

The shear flow properties (viscosity vs shear rate) of the non-crosslinked and TyrBm-crosslinked emulsions formed with pea protein alone or with zein-pea protein mixtures are shown in FIG. 15. In general, all emulsions exhibited typical shear thinning behaviour with apparent viscosity progressively decreasing with increasing shear rate indicating the presence of oil droplets in the flocculated or associated state. Shear thinning behaviour was previously reported for pea protein stabilized o/w emulsions produced at acid pH, with pectin addition, or with applied heat treatment. The apparent viscosity of both crosslinked pea protein emulsions, with or without zein addition, at the shear rate of 100 s⁻¹ was 3-fold higher than for the non-crosslinked emulsion (FIGS. 15A-B). The remarkable thickening of the TyrBm-crosslinked emulsions is consistent with the morphological observation (FIGS. 11A-B), indicating formation of a gel-like self-standing network in the system. An increase in apparent viscosity and thickening of the emulsions were observed when potato or soy protein were crosslinking by TyrBm. TyrBm induced a more evident increase in apparent viscosity when applied to a zein/potato protein mixture than to a zein/pea protein system, indicating the important influence of the water soluble protein. On the other hand, an increase of one-order of magnitude in apparent viscosity was obtained when zein was incorporated into the system, compared to emulsions stabilized by pea protein alone (FIGS. 15A-B). Additionally, the presence of zein extended the emulsion stability compared to the non-crosslinked pea protein stabilized emulsion, as has been already described and shown in FIGS. 11A-B.

The formation of the gel-like network was further confirmed by dynamic oscillatory measurement (FIGS. 17A-B). Frequency sweep tests were carried out at constant stress within the linear viscoelastic region (previously established). Progression of the storage (G′) and loss (G″) moduli with frequency for all emulsions studied is shown in FIGS. 17A-B. Non-crosslinked pea protein emulsion showed a frequency dependent behavior, while a crossover of G′ with the G″ at 0.01 Hz indicated viscous properties in nature (FIG. 17A). Both types of TyrBm-crosslinked emulsions showed frequency independent behavior followed by G′ higher than G″, presenting a predominantly elastic response and confirming a gel-like structure. TyrBm-crosslinked pea protein stabilized emulsions were characterized by 2-orders of magnitude higher G′ compared to non-crosslinked emulsions (FIG. 17A). Introducing new covalent bonds into the system led to a higher G′, which was previously observed for enzymatically crosslinked soy and potato protein stabilized emulsions and gels. Moreover, the frequency independent storage modulus is attributed to a network consisting of permanent covalent crosslinks for the enzyme induced emulsion gels, which is characteristic of classical polymer gels with permanent chemical crosslinks. When pea protein emulsion gels were formed using transglutaminase G′ values of crosslinked emulsions (14.8% w/w protein, ϕ0.1) were 1-order of magnitude higher than G′ of pea protein emulsions in our study, which could be due to a 7.4-fold higher concentration of pea protein compared to the concentration of pea protein in our emulsions. Pea protein gel-like emulsions were obtained without using enzyme, when pea protein was used in higher concentrations (above 6%, w/w, protein) with higher ϕ (0.5), by applying microfluidization (50 MPa; 1 pass; 5%, w/w, protein; 20%, w/w, oil), or when pea protein was combined with pectin.

Incorporation of zein into the system led to more pronounced gel-like behaviour of crosslinked and non-crosslinked emulsions, followed by shifting of both moduli (G′ and G″) to higher values (FIG. 17B). The non-crosslinked zein-pea protein stabilized emulsion exhibited elastic properties (G′>G′) and frequency independent behavior. Zein is known as a gelling and thickening agent, thus zein could be largely responsible for creating a gel-like protein network in the mixed emulsion gel. However, incorporation of zein during the production of non-crosslinked zein-potato protein (2% zein w/w, 6% w/w potato protein) emulsions did not have a thickening effect as high as in combination with pea protein. In addition, the non-crosslinked zein-potato protein emulsions had weaker gel with 4-fold lower G′ at 1 Hz compared to G′ values of zein-pea protein emulsions obtained in this study. These results highlight the dependence on protein type in the aqueous phase as a triggering factor in formation of a gel like structure, rather than protein concentration per se. During high pressure homogenization in non-crosslinked zein-pea protein stabilized emulsions, pea protein and zein undergo complexation, mainly maintained by non-covalent physical interactions, which can lead to formation of an elastic structure. This has been confirmed by analysis of adsorbed proteins (FIG. 12B). However, despite the fact of a higher storage than loss modulus (G′>G″) and frequency independent G′ behavior of the non-crosslinked zein-pea protein stabilized emulsion, from FIGS. 11A-B it can be observed that the emulsion was still liquid, which indicates on a natural viscous behavior. Furthermore, by introducing TyrBm into the system, the gel-like behavior was more evident and characterized by one order of magnitude higher G′ values compared to non-crosslinked zein-pea protein stabilized emulsions (FIG. 17B). These findings corroborate previous results showing that dissolving zein in the oil phase with certain plant proteins in the aqueous phase, followed by TyrBm addition, can produce significantly stronger emulsion gels with different structures depending on the water-soluble protein type.

### Example 11: Microstructure of Emulsions

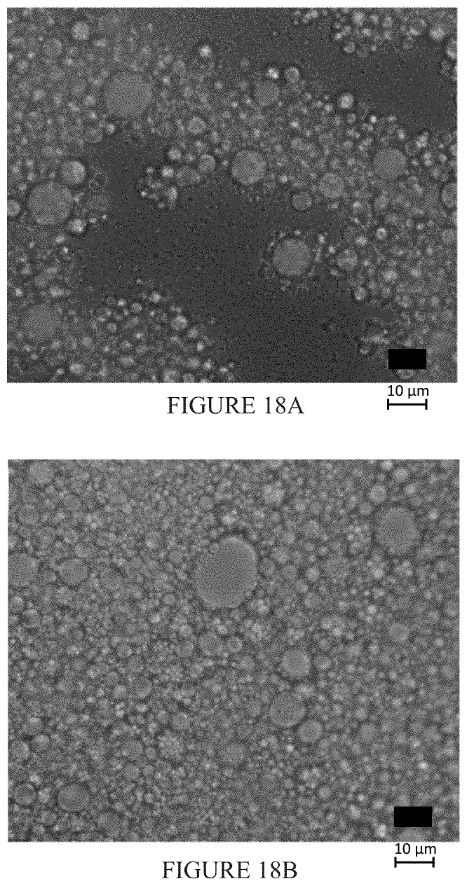

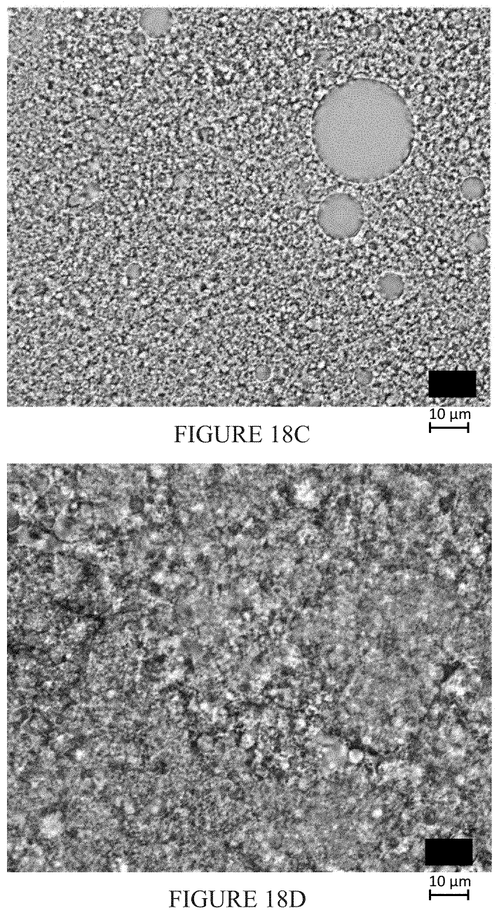

The changes in the microstructure of the produced emulsions were characterized with a Cell Observer inverted microscope, as shown in FIGS. 18A-D. The crosslinked pea protein emulsion exhibited a tighter network with close packing of oil droplets compared to non-crosslinked pea protein emulsions (FIGS. 18A, B). The significant changes in the microstructure could be observed after zein incorporation, which was characterized by very tight and dense network of oil droplets, particularly in crosslinked zein-pea protein stabilized emulsions (FIGS. 18C, D). The differences in the microstructures of the non-crosslinked and crosslinked emulsions agree with the rheological data (FIG. 17). The same findings were reported for zein-potato protein stabilized emulsions where the close packing of oil droplets was a consequence of covalent protein crosslinks formed by TyrBm, resulting in elastic gel-like appearance.

### Example 12: Examples of Crosslinked Protein-Zein Emulsions

Conditions for the preparation of stabilized protein-zein emulsions are shown in Table 4.

TABLE 4
Different conditions for the fabrication of protein-zein protein stabilized emulsions.

| | | | | | |
|---|---|---|---|---|---|
| Type of oil | Olive | Corn | Coconut | Corn | Corn |
| Type of water- soluble protein | Potato | Potato | Potato | Pea | Chickpea |
| Protein content | 6.10% | 6.10% | 6.10% | Supernatant | 2% |
| | | | | from 8% | |
| | | | | (final 2%) | |
| Ratio of ethanol to oil | 10% | 10% | 10% | 10% | 10% |
| Zein content | 2% | 2% | 2% | 2% | 2% |
| Ratio of oil to water phase | 40% | 40% | 40% | 40% | 20% |
| Shear stress | 35000 rpm/ | 35000 rpm/ | 35000 rpm/ | 3 passes, | 4 passes, |
| | 5 min | 5 min | 5 min | 20 kPsi | 20 kPsi |
| Tyrosinase | TyrBm | TyrBm | TyrBm | TyrBm | TG TI |
| Crosslinking enzyme content | 0.033 (1:30) | 0.033 (1:30) | 0.033 (1:30) | 0.04 | 150 U/g |
| (per g water soluble protein) | | | | | |
| Temp. (C.) | 37 | 37 | 37 | 37 | 37 |
| Crosslinking time (h) | 4 h | 4 h | 4 h | 2 h | 4 h |
| Stirring (rpm) | 220 | 220 | 220 | 100 | no |
| pH | 7 | 7 | 7 | 10 | 10 |
| Ionic strength | 25 mM | 25 mM | 25 mM | 50 mM | 50 mM |

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound comprising (i) a zein protein having a first moiety, and (ii) a polypeptide of a protein extract, said polypeptide having a second moiety, wherein said first moiety and said second moiety are linked to each other.

2. The compound of claim 1, wherein said first moiety and said second moiety are linked to each other via a covalent bond, optionally wherein said zein protein is selected from the group consisting of: alpha-zein, beta-zein, gamma-zein, and delta-zein.

3. The compound of claim 1, wherein at least one moiety selected from said first moiety and said second moiety is a tyrosine moiety.

4. The compound of claim 1, wherein at least one moiety selected from said first moiety and said second moiety is selected from the group consisting of: lysine, tyrosine and cysteine.

5. (canceled)

6. A composition comprising a plurality of compounds according to claim 1, wherein said plurality of said compounds is in the form of an agglomerate of particles.

7. The composition of claim 6, wherein at least 25% of said plurality of said compounds are characterized by a size in the range of 15 micrometers to 350 micrometers.

8. The composition of claim 6, being in the form of oil-in-water emulsion, optionally wherein said emulsion comprises 5% to 50% (w/w) oil.

9. (canceled)

10. The composition of claim 6, being in the form of a gel or a paste and/or being characterized by a storage modulus of at least 200 Pa.

11. (canceled)

12. (canceled)

13. The composition of claim 6, comprising an oil-in-water emulsion, wherein said emulsion comprises 1% to 80% (w/w) of oil, by weight.

14. The composition of claim 13, wherein said oil is selected from olive oil, corn oil, coconut oil, or a combination thereof.

15. The composition of claim 13, further comprising a cross-linking enzyme, and optionally an enzymatic cross-linking mediator.

16. The composition of claim 15, wherein said enzyme is tyrosinase, optionally wherein said tyrosinase is derived from Bacillus megaterium.

17. (canceled)

18. The composition of claim 16, wherein said enzyme is characterized by an enzymatic activity in a condition selected from: pH in the range of from 5 to 11.5, and a temperature in the range of from 20 to 65° C.

19. The composition of claim 6, wherein a concentration of said zein protein is in the range of from 0.1% to 10% (w/w), a concentration of said polypeptide of said protein extract is in the range of from 1% to 25% (w/w) and a concentration of said tyrosinase in the range of from 0.05% to 0.5% (w/w).

20. The composition of a claim 16, wherein said tyrosinase and said polypeptide of said protein extract are in a ratio ranging from 1:20 to 1:200.

21. (canceled)

22. A process for covalently linking a zein protein and a polypeptide of a protein extract, the process comprising the steps of:
(a) mixing a solution comprising a zein protein, a solution comprising polypeptide of a protein extract, and a solution comprising tyrosinase thereby creating a mixture; and
(b) incubating said mixture for at least 1 h at 25-60° C., thereby covalently linking said zein protein and said polypeptide of said protein extract.

23. The process of claim 22, wherein any one of: (i) said first solution comprises from 0.2% to 20% of zein protein, by weight, and 1% to 50% ethanol, by weight, and wherein said second solution is an aqueous solution comprising 6% to 40%, by weight, of said polypeptide of said protein extract; (ii) said first solution comprises from 0.2% to 20% of zein protein, by weight, 0.2% to 50% ethanol, by weight, and 1% to 80% oil, by weight, and wherein said second solution is an aqueous solution comprising 0.1% to 40%, by weight, of said polypeptide of said protein extract; and (iii) said oil selected from olive oil, corn oil, coconut oil, or any combination thereof.

24. (canceled)

25. (canceled)

26. A compound comprising a polypeptide derived from a zein protein and an organic molecule, wherein: (i) said organic molecule comprises a moiety selected from the group consisting of: primary amine, phenol and thiol, or any combination thereof, (ii) said polypeptide and said organic molecule are linked to each other via a covalent bond comprising:
(a) a phenolic moiety, and
(b) another moiety selected from the group consisting of: primary amine, phenol, or thiol.

27. The compound of claim 26, wherein said organic molecule is selected from the group consisting of: proteins, polysaccharides, polynucleic acids, and a therapeutic agent.

28. (canceled)
